(19) United States
(12) Patent Application Publication
(10) Pub. No.: US 2024/0335617 A1
(43) Pub. Date: Oct. 10, 2024

(54) ADMINISTRATION DEVICE WITH DETACHABLE INDICIUM

(71) Applicant: AKTIVAX, INC., Broomfield, CO (US)

(72) Inventors: Amir Genosar, Broomfield, CO (US); Jason Fox, Greenwood Village, CO (US)

(21) Appl. No.: 18/293,262

(22) PCT Filed: Jul. 28, 2022
(86) PCT No.: PCT/US2022/038702

§ 371 Date: Jan. 29, 2024

Related U.S. Application Data
(60) Provisional application No. 63/227,220, filed on Jul. 29, 2021.

(51) Int. Cl. A61M 5/315; A61M 5/20; A61M 5/32

(52) U.S. Cl. CPC A61M 5/31568; A61M 5/20; A61M 5/3202

(57) ABSTRACT

A beneficial agent dispensing device system is provided. The beneficial dispensing device system includes a dispensing device containing a beneficial agent, an indicium coupled to and selectively decouplable from the dispensing device, the indicium indicating information related to the beneficial agent, and a securing mechanism coupled to the indicium and configurable to secure the indicium to a securable feature.

### BACKGROUND

The tracking of the administration of medicaments is important in a number of settings. For instance, in medical settings, the tracking of medicaments administered to patients is important for maintaining the health of the patients and limiting malpractice liability. In many settings, tracking of medicaments administered to patients can be accomplished with people consistently entering data into computing devices, perhaps at multiple points between the acquiring of the substances and the administration of the substances. This system can be effective, but it also lends itself to human error. For instance, the person having administered the substance may expect an accompanying person to document the administration, and the accompanying person may not recognize the implicit obligation. Further, an entry made can be in error. For instance, the medication to be injected may have labels on bottles, but an entry of the data representing the drugs may be in error. Further, the identities of patients to whom the substances are administered may be in error.

In some instances, the issues can be resolved by checking multiple points of data entry, but even in those situations, it may be difficult to resolve which conflicting source is correct. Also, in medical settings, getting access to data devices that track the information may be difficult or cumbersome, for instance, in an emergency setting. Further, there exists the possibility that a health professional may forget whether the drug has been administered and to whom the drug has been administered in a room with multiple patients. Attempting to physically label individual patients to discern between the patients may be cumbersome without a convenient method to track which drug corresponds to a label and then to a patient. This may be particularly evident in a triage or emergency situation, in battlefield conditions, or in other chaotic environments in which a medicament may be administered. Such physical labels may also suffer from the same limitations as the aforementioned data entry, with the labels not being specifically associated with any particular drug or patient. Further, the labels may be difficult to arrange and dispense when necessary.

### SUMMARY

The described technology provides implementations of a beneficial agent dispensing device system. The beneficial dispensing device system includes a dispensing device containing a beneficial agent, an indicium coupled to and selectively decouplable from the dispensing device, the indicium indicating information related to the beneficial agent, and a securing mechanism coupled to the indicium and configurable to secure the indicium to a securable feature.

An example method for tracking a dispensable substance in a dispensing device in accordance with the presently disclosed technology includes decoupling an indicium from the dispensing device and coupling a securing mechanism attached to the indicium to a securable feature.

This summary is provided to introduce a selection of concepts in a simplified form that is further described below in the Detailed Description. This summary is not intended to identify key features or essential features of the claimed subject matter, nor is it intended to be used to limit the scope of the claimed subject matter.

Other implementations are also described and recited herein.

### BRIEF DESCRIPTIONS OF THE DRAWINGS

FIG. 1 illustrates an example of a beneficial agent dispensing system for administering and tracking an administration of a substance.

FIG. 2A illustrates an example of the beneficial agent dispensing device system in an initial safe configuration.

FIG. 2B illustrates an example of a decoupling configuration in which the indicium with the securing mechanism decouples from the dispensing device.

FIG. 2C illustrates an example of a decoupled configuration in which the cap is decoupled from the dispensing device

FIG. 2D illustrates an example of an engaged configuration in which the securing mechanism is engaged with an anatomical feature of a person.

FIG. 2E illustrates an example of a securing configuration in which the securing mechanism secures the anatomical feature.

FIG. 2F illustrates an example of a secured configuration in which an anatomical feature is secured by the securing mechanism.

FIG. 3A illustrates an example of a securing mechanism with a wristband at a distal end of a band relative to the cap.

FIG. 3B illustrates an example of a securing mechanism with a wristband formed between the coupler and the cap.

FIG. 3C illustrates another example of a securing mechanism with a wristband at a distal end of a band relative to the cap.

FIG. 3D illustrates an example of a securing mechanism in which a portion of a band is coupled to another portion of a band.

FIG. 4A illustrates a top view of an example of a system with a cap including holes.

FIG. 4B illustrates a side view of an example of a system for coupling the cap to the indicium.

FIG. 4C illustrates another side view of an example of a system for coupling the cap to the indicium.

FIG. 4D illustrates still another side view of an example of a system for coupling the cap to the indicium.

FIG. 5A illustrates an example system with a retractable lanyard.

FIG. 5B illustrates an example system with a carabiner-type loop directly coupled to the cap.

FIG. 5C illustrates an example system with an indicium that includes a lanyard integrated into a cap.

FIG. 5D illustrates an example system with an indicium that includes a cable with a threaded connector.

FIG. 6 illustrates an example of a cap.

FIG. 7A illustrates an example of an arrangement of an autoinjector.

FIG. 7B illustrates a cross-section of an example of an autoinjector in a first autoinjector state.

FIG. 7C illustrates an example of an autoinjector in a second autoinjector state and a mixing autoinjector state.

FIG. 7D illustrates an example of an autoinjector in an armed state.

FIG. 7E illustrates an example of an autoinjector in a manufacturing configuration.

FIG. 8A illustrates an example dispensing device system with a dispensing device in a prearmed state before tearing of a label.

FIG. 8B illustrates an example dispensing device system with a dispensing device on which a securing mechanism has torn through a label comprising the indicium.

FIG. 8C illustrates an exploded view of an example dispensing device system.

FIG. 8D illustrates an example dispensing device system with a dispensing device.

FIG. 8E illustrates an example dispensing device system in a fully torn and deployed configuration.

FIG. 9A illustrates an example of a dispensing device in an unarmed state in which an indicium is coupled by a cap to the dispensing device.

FIG. 9B illustrates an example of a dispensing device in an armed state in which the cap is removed, decoupling the indicium from the dispensing device.

FIG. 10 illustrates a flowchart of an example method for tracking a dispensable substance in a dispensing device comprising a decouplable indicium.

FIG. 11 illustrates a flowchart of an example method for tracking a dispensable substance in a dispensing device comprising a decouplable indicium.

FIG. 12 illustrates a flowchart of an example method for tracking a dispensable substance in a dispensing device comprising a decouplable indicium.

### DETAILED DESCRIPTIONS

Systems and methods for activating containers and tracking the contents of containers are disclosed. Containers and their contents can be difficult to track in certain circumstances. For instance, in medical environments, drug containers must be carefully handled and tracked to assure that the correct substances are administered to the correct people. In most systems, there are at least a few points where human error can defeat tracking of the substances from storage to patient.

A common type of beneficial agent administering device or dispensing device is a prefilled injection device, for instance, an autoinjector. The prefilled injection device may be for parenteral drug delivery. The dispensing device may include a body in which a dispensable substance, such as a beneficial agent (e.g., a drug or other medical composition), is contained. The system may further include a cap coupled to the dispensing device. The cap may be such that the substance cannot be removed or administered from the prefilled injection device without first removing the cap. The cap may be attached to an indicium indicating at least the identity of a dispensable substance in the injector. The indicium may also be removably coupled to a body of the dispensing device, such that the indicium can be removed from the body of the dispensing device when the cap is removed from the injector. The body may also be described as a shell.

The indicium may also include or be coupled to a securing mechanism with which the indicium may be selectively engaged at the discretion of a user to secure the indicium to a securable feature. A securable feature can include a feature of one or more of a patient (e.g., an appendage or other portion of a person or animal), an article of clothing worn by the patient, a bedpost or rail of a bed occupied by the patient, or another element in the proximity of the patient (e.g., in a patient environment) or the equivalent of the patient to whom or which the dispensable substance is administered. The securing mechanism may form a non-reversible attachment to the securable feature, meaning the securing action may not be de-secured or disconnected by simple reverse action of the same mechanism. For example, the non-reversible attachment may be removed from the patient after the securing mechanism is coupled to the patient (e.g., exclusively) by destroying or otherwise modifying the non-reversible attachment in a manner in which it is apparent that the non-reversible attachment has been severed. For example, the non-reversible attachment may include an irreversible zip tie or cable tie.

The indicium and/or the securing mechanism may include elements specific to coupling the indicium to the patient. For instance, a band or lanyard. In implementations, the securing mechanism comprises a band or lanyard adapted for selective engagement with a securable feature. The indicium may comprise a modifiable portion suitable to indicate further information related to the stored agent, such as administration time, patient name, dose, or other information. The modifiable portion may comprise a writable section, a section on which a separate label may be affixed, a section comprising punch-out alphanumeric characters, or equivalent structures. The band or lanyard may be configured to be adjustable to facilitate coupling to patients of different sizes. For instance, the lanyard may include a mechanism by which the indicium is tightened around a patient's anatomical feature, for instance, a patient's appendage such as a wrist, ankle, or the like. The cap may be an element of or attached to the indicium that remains coupled to the indicium after the injector is discarded, the cap potentially aiding in facilitating the tightening of elements of the securing mechanism coupled to the indicium. In an implementation, the securing mechanism and/or the indicium or a lanyard thereof may include a removable portion of a label (e.g., a label adhered to or integral to the dispensing device).

A cap may be coupled to the device in a safe configuration and decoupled from the dispensing device in an armed configuration. In implementations, the indicium is coupled to the cap in the safe configuration but is separable in the armed configuration. In other implementations, the indicium maintains an attachment to the dispensing device in both the safe and armed configurations.

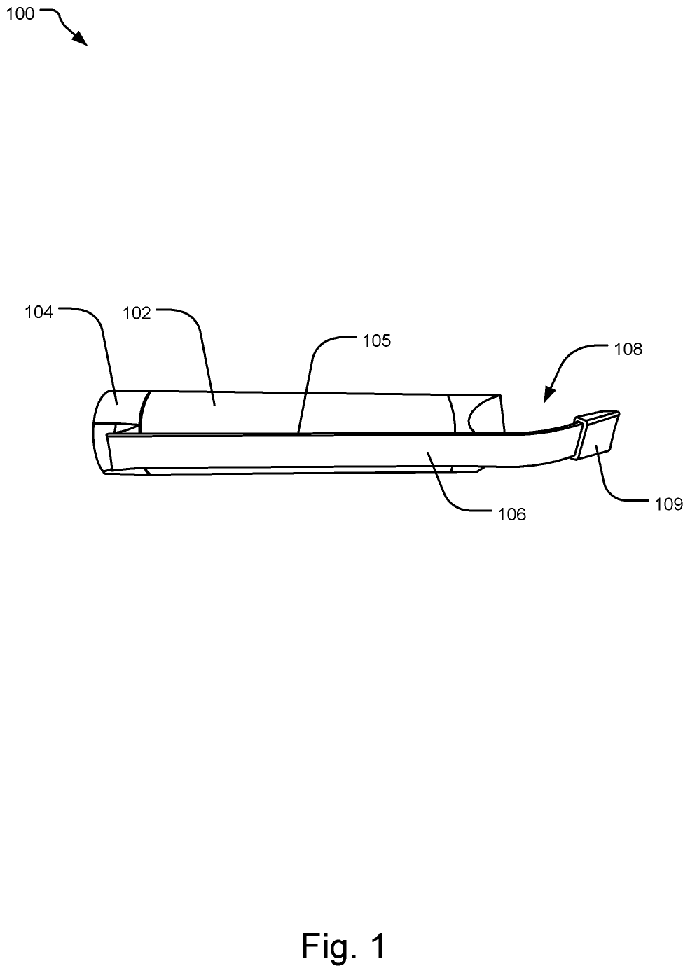

FIG. 1 illustrates an example of a beneficial agent dispensing system 100 for administering and tracking an administration of a substance. The dispensing device 102 is a device that dispenses a dispensable and/or administrable substance, such as a beneficial agent or medicament, stored in the dispensing device 102. The term dispensing device may also be referred to as an administration device. In an embodiment, the dispensing device 102 may be an injector, such as an autoinjector, or any administration or dispensing device. The dispensing device may be operable to inject the dispensable substance into a patient. The dispensing device 102 may be coupled to and selectively decoupled from a cap 104. The cap 104 may be a cap of the dispensing device 102 that may be decoupled from the dispensing device 102. In implementations, in a safe configuration, the cap 104 may prevent dispensing of the dispensable substance from the dispensing device 102 prior to removal of the decouplable cap 104 from the dispensing device. That is, removal of the cap 104 may coincide with or otherwise cause a transition between the safe configuration and an armed configuration. The transition may include “arming” or making the dispensing device 102 ready for injection in an armed configuration. The dispensing device 102 may be activated or armed when the cap 104 is removed simply by exposing the elements of the dispensing device 102 that administer or otherwise dispense the dispensable substance. Alternatively or additionally, the dispensing device 102 may include activating elements, for example, one or more of a retainer, safety pin, and safety catch, and the activating elements may be functionally coupled or couplable to the cap 104. In this example, removing the cap 104 may actively remove or deactivate safety elements and facilitate administration and/or dispensing of the dispensable substance to place the dispensing device in an armed dispensing device state. The cap 104 may be attached or separably or fixedly coupled to an indicium 106.

The indicium 106 may include information related to the dispensable substance, including, for example, information associated with one or more of the dispensing device 102, the dispensable substance contained in the dispensing device 102, instructions for use (IFU) of the dispensing device 102, and/or a person to whom the dispensable substance may be administered. The indicium 106 may be coupled to and selectively decouplable from a portion of the dispensing device 102, such as a body portion of the dispensing device 102, in addition to being coupled to the cap 104. For example, the indicium 106 may be a portion of a label that is selectively detachable or decouplable from the dispensing device 102, such as by tearing a perforation 105 on a portion of a label. In the illustrated example, the indicium 106 is depicted comprising a pull tab 109 or pull, which a user can pull to cause the tearing or decoupling. The indicium 106 may be an element of a label or may be situated between a body or shell of the dispensing device 102 and a label prior to the decoupling of the indicium 106. The indicium 106 may be an element of a label that is perforated and easily decouplable (the perforations to guide the tearing) or may be situated under a label including a perforated portion prior to the decoupling of the indicium 106. The indicium 106 and/or a securing mechanism 108 may provide a pull or pull tab to remove an activation member or element of the dispensing device 102. The activation member may be configured so that the activation member's removal will cause the dispensing device 102 to transition to an armed state.

The indicium 106 may initially be further coupled to the dispensing device 102 indirectly via the coupling of the indicium 106 to the cap 104. Moreover, the indicium 106 may include an indication of when the substance of the dispensing device 102 has been dispensed and/or administered. In an implementation, removal of the indicium 106 from the body portion or a separate manual removal of a portion of the indicium 106 may expose an active timing element (e.g., a pressure-sensitive, photosensitive, or chemosensitive element) that “develops” upon exposure to light or environmental air to provide an indication of an elapsed time since the indicium 106 or element was removed and/or the substance was administered or otherwise dispensed. Alternatively or additionally, the indicium 106 may feature an area adapted to be marked by a user to indicate administration time. Alternatively or additionally, an indication of a time of use may be by writing information in a designated area or making a physical change to the indicium which doesn't require a writing instrument, such as scratching, peeling, tearing, or perforating (e.g., by punching a hole next to a selection).

The indicium 106 may be further coupled to or include a securing mechanism 108. The securing mechanism 108 is a mechanism used to secure the indicium 106 to a person. The securing mechanism 108 may be configurable or configured to secure the indicium to a securable feature, such as an anatomical feature of the person, for instance, an appendage of the person or an element of a patient environment. The securing mechanism 108 may be coupled irreversibly to a person, for instance, not allowing removal after the securing mechanism is secured without damaging the securing mechanism 108. In implementations, the decoupling of the indicium 106 from the dispensing device 102 may expose the securing mechanism, allowing the securing mechanism 108 to be secured to the securable feature.

In various implementations, the indicium 106 may be coupled to the cap 104 and the dispensing device 102. The cap 104 may be coupled to the dispensing device in a manner that prevents the dispensing device from dispensing. For example, the cap 104 and/or the dispensing device 102 may include arming elements that can arm the dispensing device 102 responsive to the cap 104 being removed. Decoupling of the indicium 106 from the dispensing device 102 may cause the cap 104 to decouple from the dispensing device 102, and, consequently, cause the arming elements in one or more of the cap 104 and the dispensing device 102 to arm the dispensing device 102. In the unarmed state (first or unarmed configuration), the dispensing device 102 may be prevented from administering or prevent the administration or dispensing of an administrable substance. In an armed state (second or armed configuration), the dispensing device is able to administer or is allowed to administer or dispense the administrable substance. In implementations in which the indicium 106 is coupled to both of the cap 104 and the dispensing device 102 at different locations, the decoupling of the indicium 106 from the dispensing device 102 at a location (e.g., by tearing through a label that couples the indicium 106 to the dispensing device 102) may or may not be sufficient to cause the cap 104 to be removed (and arming of the dispensing device 102). In this implementation, a further pull on the indicium 106 with a certain predefined threshold force removes the cap 104 to arm the dispensing device 102.

Implementations are contemplated in which the dispensing device 102 includes a strap that is not configured to couple to an anatomical feature of a patient (e.g., that the securing mechanism 108, as illustrated, is just a strap or band (e.g., an element of a lanyard) that is not for coupling to a patient). For example, the indicium 106 may be coupled to the cap 104, or the indicium 106 may not be related to a specific patient. For example, in implementations where the indicium 106 includes instructions for use but does not include patient-specific information, there may be no need to couple the indicium 106 to the patient. Implementations are also contemplated in which the securing mechanism 108 is for securing the indicium 106 to things other than a patient, for example, a bed, an article of clothing, or a vehicle.

In an implementation, the securing mechanism 108 may include a band and a cinch coupled to the band. The cinch may be irreversibly advanceable along at least a portion of the band. The cinch may be situated on the band relative to the cap 104, such that pulling the cap 104 when the band is engaged to an anatomical feature of a person tightens the band about the anatomical or other securable feature of the patient. In one implementation, the band is coupled at both a first end of the band and a second end of the band to the cap 104, forming a band loop. The band may be threaded twice through the cinch. The securing mechanism 108 may receive an anatomical feature of the person between the cinch and the band loop. The securing mechanism 108 may then be secured to the anatomical feature by irreversibly advancing the cinch distally from the cap 104. In various implementations, the cinch is stored in or adjacent to the cap or in or adjacent to an endpiece prior to the decoupling of the indicium 106 and/or cap 104 from the dispensing device 102. The cinch may be disposed at a terminal end of the band prior to the decoupling of the indicium from the dispensing device 102. In some implementations, the band may be decouplable from the cap 104.

In another implementation in which the band is coupled at both a first end of the band and a second end of the band to the cap 104, forming a band loop, the securing mechanism 108 receives an anatomical or other securable feature of the patient between the cinch and the cap 104. The securing mechanism 108 may then secure the anatomical feature by irreversibly advancing the cinch proximally towards the cap 104.

In implementations in which a loop is formed by the band of the securing mechanism 108, the securing mechanism may include an endpiece on a distal end of the band, perhaps limiting the motion of the cinch.

In an alternative implementation, the band is coupled at a first end of the band to the cap 104 and a second end of the band to the cinch. In this implementation, the band may be threaded once through the cinch, forming a band loop. The securing mechanism 108 may receive an anatomical feature of the person between the cinch and the band loop. The securing mechanism 108 may then be coupled to the anatomical feature by irreversibly advancing the cinch distally from the cap 104.

In still another alternative implementation, two bands may be coupled to the cap 104, or a band may be threaded through the cap 104 to effectively make two separate bands. The second of the bands may include a coupling element, such as an adhesive. An anatomical feature may be placed between the bands, and the adhesive on a second band may be used to couple (e.g., adhere) the portion of the second band with the coupling element to a portion of the first band. This may effectively secure the anatomical feature between the coupled bands. In an implementation in which the two bands are defined by a single band having been threaded through the cap 104, the band lengths may be adjusted by pulling the band through the cap to a desired length. This may allow for the coupling between the band portions (in this instance, on the same band) to more effectively secure the anatomical feature of the person.

In various embodiments, the securing mechanism 108 may include a wristband that is attached to the cap 104. For example, the wristband can be made of a stretchable material that can be expanded while placing the wristband on the anatomical feature of a person. Other types of wristbands, such as those commonly used with wristwatches, are also contemplated. Such wristbands may include a ratchet mechanism like a zip-tie or clasp mechanism. Another type of wristband contemplated is a flexible spring band, perhaps a flexible stainless steel bistable spring band (e.g., a slap bracelet or snap bracelet). These bistable spring bands may be covered with or encased by a material more comfortable for a person's appendage, for example, one or more of fabric, silicone, or plastic. The indicium 106 may comprise a pin or a clip for creating a loop or attaching to a subject or an object. The indicium may be configured to form a tie to create a loop or to secure the indicium to a subject or an object.

During storage, the wristband may be retained under a label and against a shell or body of the dispensing device 102, which may be an autoinjector. The label may include a perforation 105 along its length on either side or both sides of the wristband of the indicium 106. Decoupling the indicium 106 and its securing mechanism 108 from the dispensing device may tear through the label and potentially expose a clear shell of the dispensing device. During arming of the dispensing device to allow for dispensing of a dispensable substance, a user may pull down on a looped end of the wristband, breaking the labels and tearing the wristband away from the shell of the dispensing device, effectively decoupling the indicium 106 and the securing mechanism 108 away from the body or shell of the dispensing device 102. Once completely detached from the shell of the dispensing device, the user may pull on the wristband of the securing mechanism 108 to remove the cap 104 and arm the dispensing device 102. The removed section of the label may then expose a clear shell of the dispensing device 102 underneath the label to reveal the dispensable substance contained in the dispensable device and allow a person to verify that the dispensable substance has been dispensed after activation of an active indication element after arming or administration. Alternatively, the removed section of the label may expose a second label portion. The label portion may include a time indicator or instruction for use after arming.

Implementations are contemplated in which the cinch is stored in the cap 104 prior to removal of the cap 104 from the dispensing device 102.

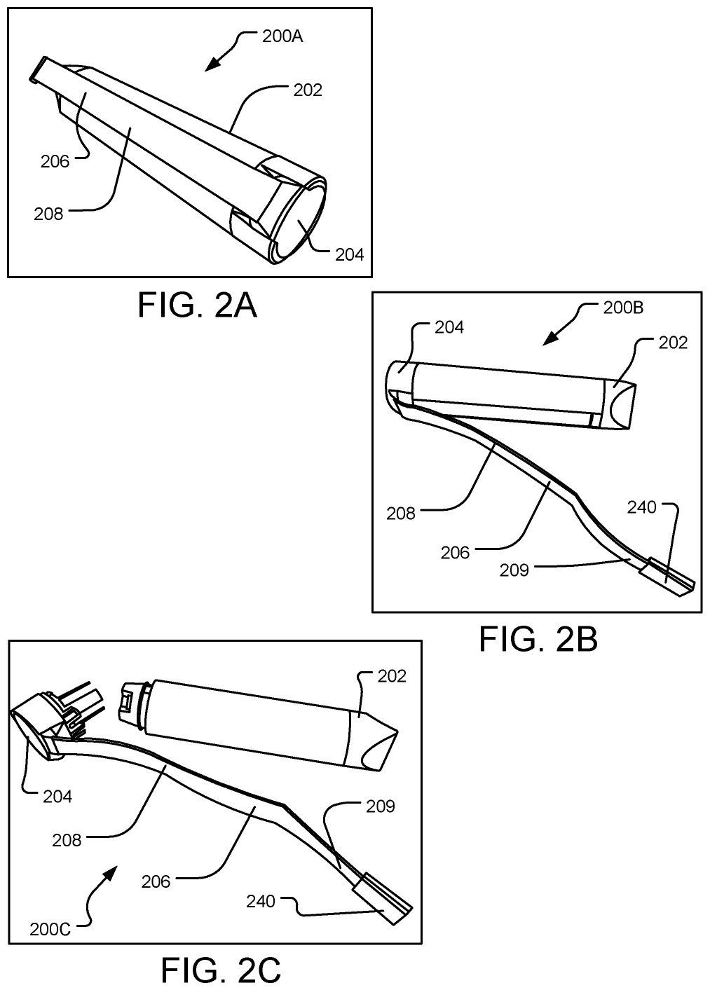

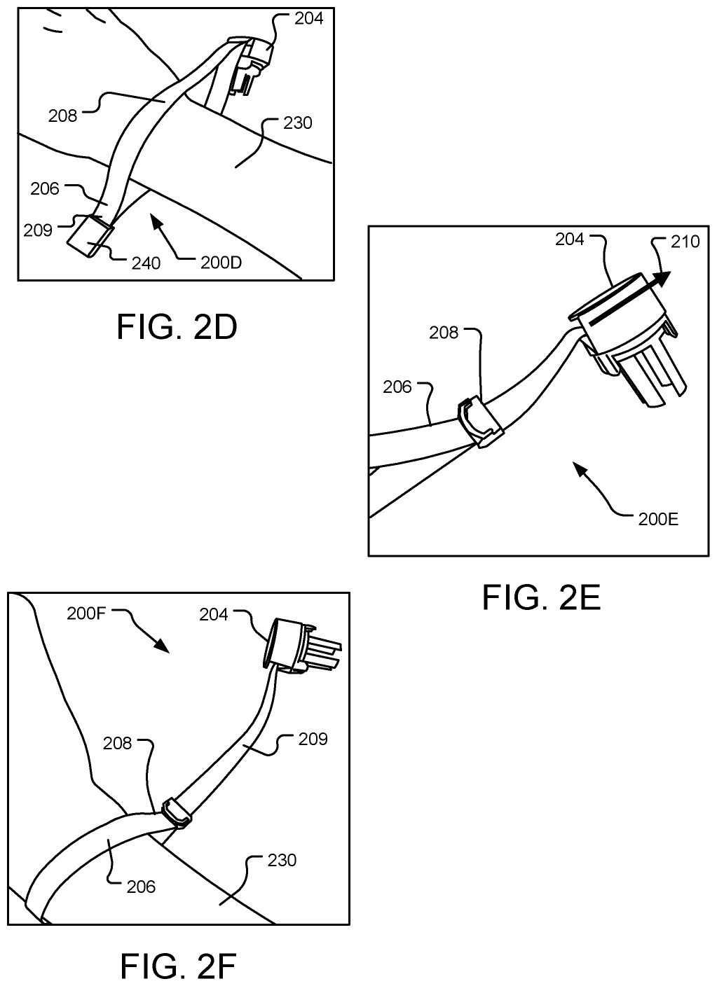

FIGS. 2A-2F illustrate an example of a progression of using a substance dispensing system. Dispensing device 202, cap 204, indicium 206, and securing mechanism 208 may comprise implementations of dispensing device 102, cap 104, indicium 106, and securing mechanism 108, respectively. FIGS. 2A-2F may represent a self-cinching implementation. Self-cinching may involve a wristband that is coupled to itself, so pulling one end of the wristband may pull the cinch toward a user's anatomical or securable feature, such as a wrist. Self-cinching implementations may only require one hand to operate, perhaps allowing the person to whom the indicium is secured to self secure the indicium without assistance. In an implementation, one end of a wristband of the securing mechanism 208 may be attached securely to the cap 204. The other end may be attached securely to a cinch. Both sides of the wristband may run through the cinch. The cinch may be housed in the cap during storage. An end piece may be attached securely to a looped end of the wristband for gripping and to ensure the cinch does not pull out of the cap 204 during arming.

Specifically, FIG. 2A illustrates an example of a beneficial agent dispensing device 202 in an initial safe configuration 200A. The dispensing device 202 has the cap 204 coupled to the dispensing device 202 at an end. The indicium 206 is depicted in a variant comprising a pull tab 209 or pull attached or coupled to an endpiece 240, with the securing mechanism 208 coupled by a perforated coupling or by a label to the dispensing device 202.

FIG. 2B illustrates an example of a decoupling configuration 200B in which the indicium 206 with the securing mechanism 208 decouples from the dispensing device 202. The decoupling may occur along the perforated coupling and/or through tearing a label. In this regard, the indicium 206 may comprise a strip of material detachably attached to the dispensing device 202. In other examples, the indicium 206 may comprise a strip of material defined between two perforations of a film, wrap, or other material that may extend relative to the dispensing device 202 (e.g., in some examples, may extend about the entirety of the dispensing device). The indicium 206, as illustrated, includes an endpiece 240 joined to a pull-tab 209 that can be used to aid in the decoupling. Also shown in FIG. 2B, removal of the indicium 206 may expose a clear portion of the dispensing device 202. This may allow a user to view and verify that the substance to be administered is present in the dispensing device 202. This may also allow a user to determine if the dispensing device 202 has been used or is spent.

FIG. 2C illustrates an example of a decoupled configuration 200C in which the cap 204 is decoupled from the dispensing device 202. The decoupling of the cap 204 from the dispensing device 202 may expose elements that allow dispensing of the dispensable substance from the dispensing device 202. Further, specific coupling elements between the cap 204 and the dispensing device 202 may activate administering or dispensing elements in the dispensing device 202 when the cap 204 is decoupled from the dispensing device 202 to arm the dispensing device 202.

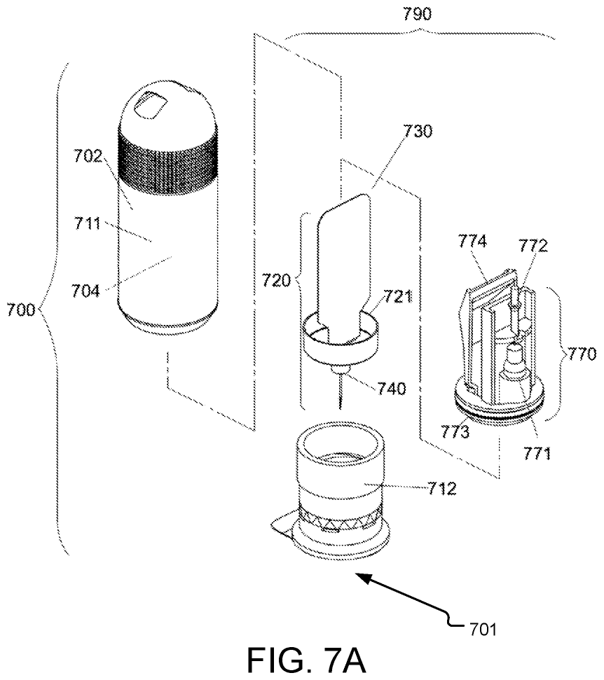

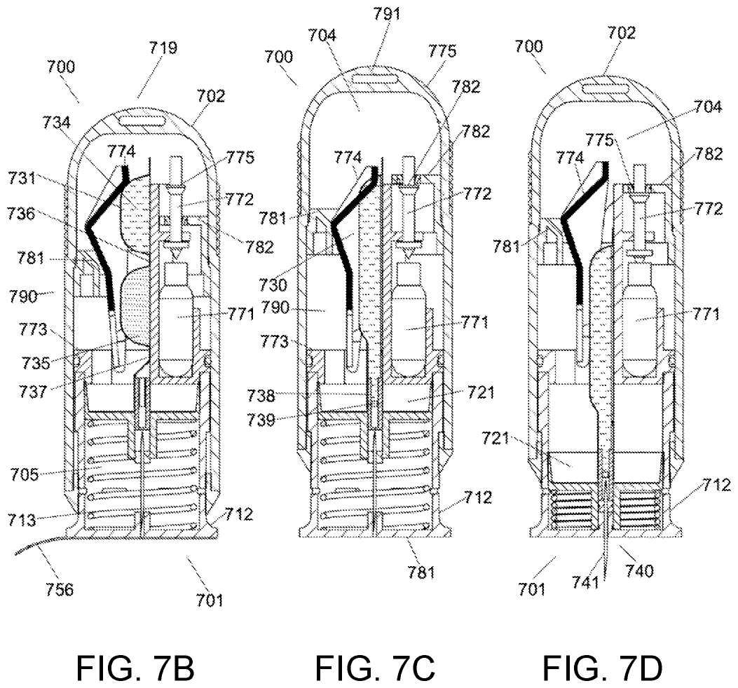

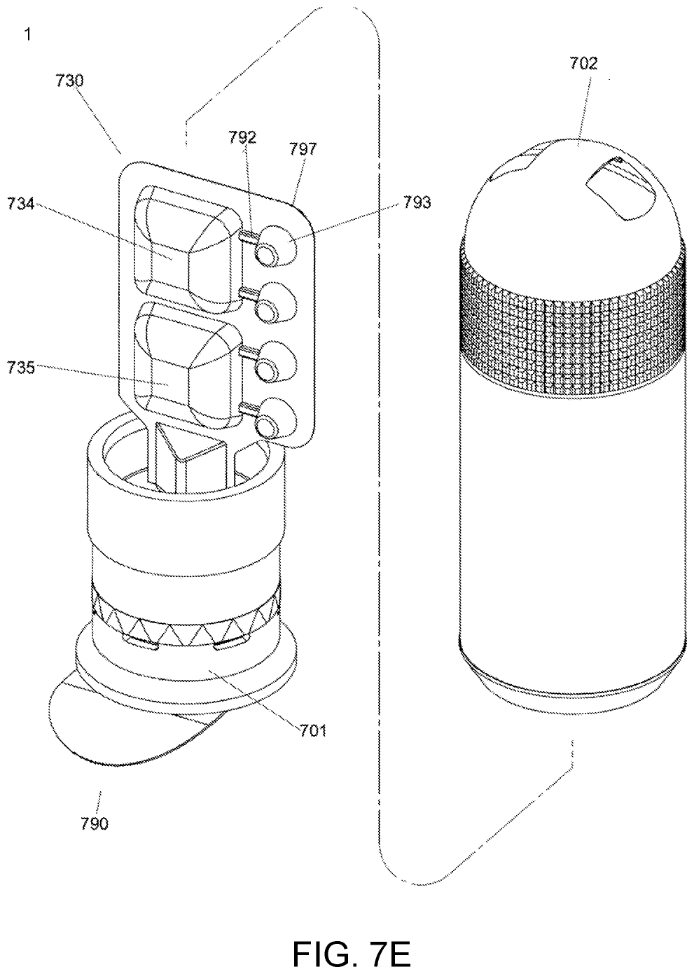

FIGS. 2A-2C demonstrate a progression of the dispensing device 202 transitioning from an unarmed state (or first configuration or safe configuration) to an armed state (or second configuration or armed configuration). In the illustrated implementation, when the cap 204 is coupled to the dispensing device 202, the dispensing device may be in an unarmed state. In the unarmed state, the dispensing device 202 may be incapable of dispensing the dispensable substance in the dispensing device 202. An example implementation of an arming mechanism is illustrated in FIGS. 7A-7E. In the unarmed state (first or safe configuration), the dispensing device 202 may be restricted or prevented from dispensing the dispensable substance (e.g., a beneficial agent or administrable substance) in the dispensing device 202. In the armed state (second or armed configuration), the dispensing device 202 may enable dispensing of the dispensable substance. The transition from the unarmed state to the armed state may be facilitated by arming elements of the dispensing device 202. These arming elements may be triggered to arm the dispensing device upon a triggering event (e.g., one or more of removing a cap 204, an indicium 206, and/or a securing mechanism 208).

FIG. 2D illustrates an example of an engaged configuration 200D in which the securing mechanism 208 is engaged with an anatomical feature 230 of a person. In this example, the anatomical feature 230 is an arm of a person. In this example, the anatomical feature 230 is inserted between a cinch originally stored in the cap 204 and an endpiece 240 joined to a band of the indicium 206 comprising pull-tab 209.

FIG. 2E illustrates an example of a securing configuration 200E in which the securing mechanism secures the anatomical feature 230. In this implementation, the cinch is coupled to one end of the band with the cap 204 coupled to the other end of the band of the securing mechanism 208. The arrow 210 illustrates that the cap 204 may be pulled to advance the cinch away from the cap and to secure the anatomical feature 230 between the cinch and a loop of the band.

FIG. 2F illustrates an example of a secured configuration 200F in which an anatomical feature 230 is secured by the securing mechanism 208. The indicium 206 comprising a pull-tab 209 joined to an endpiece 240 may be seen displaying information, while the securing mechanism 208 is secured to the anatomical feature 230. In an alternative implementation, the indicium 206 omits a distinct tab or endpiece. The indicium 206 may visibly display information while the securing mechanism 208 is secured to the anatomical feature 230.

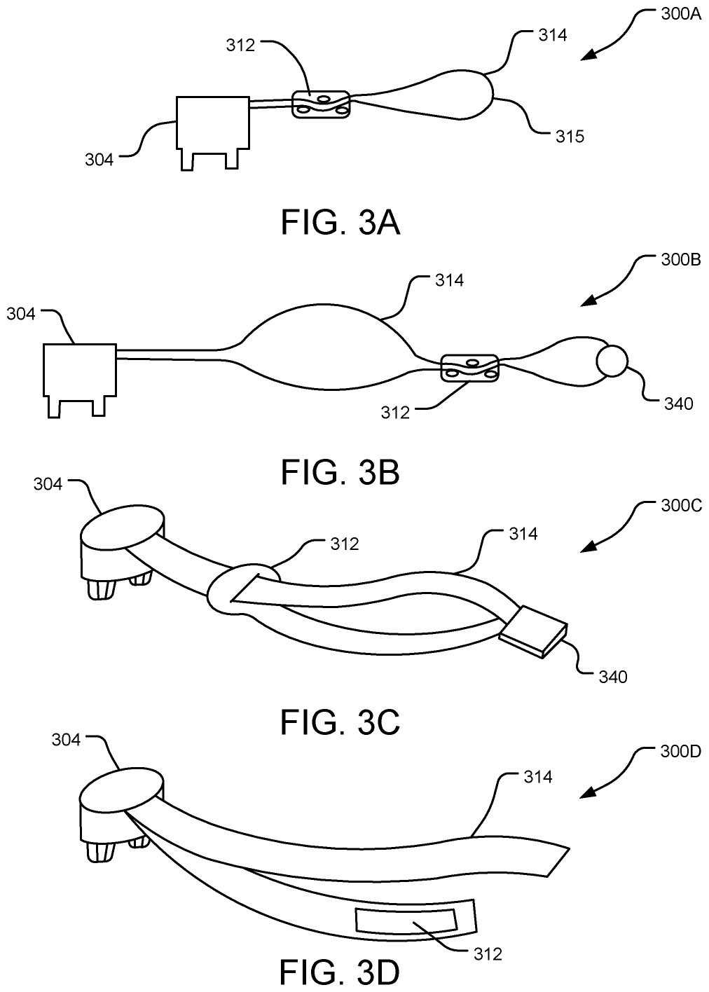

FIGS. 3A-3D illustrate examples of the securing mechanism 300A-300D. The securing mechanisms 300A-300D may be implementations of securing mechanism 108. The securing mechanisms 300A-300D may comprise one or more of a cap 304, a coupler 312, a band 314, and an endpiece 340. The cap 304 may be an implementation of the cap 104.

Specifically, FIG. 3A illustrates an example of a securing mechanism 300A with a wristband at a distal end of a band 314 relative to the cap 304. The securing mechanism 300A includes a coupler 312 that is a cinch. The coupler 312 has the band 314 threaded through twice. The band 314 is coupled to the cap 304 at both ends. The securing mechanism 300A receives an anatomical feature in a wristband formed between the coupler 312 and a bend 315 of a loop of the band 314. The bend 315 is at a terminal end of the loop when the band 314 is pulled the furthest from the cap 304 at the terminal end, causing the sides of the loop to meet. The coupler 312 can be advanced distally from the cap 304 to secure the anatomical feature. The securing mechanism 300A may be an implementation of a non-self-cinch concept. For the purposes of this specification, a non-self-cinch may include two handed operation. Therefore, operating the securing mechanism 300A may involve one hand to hold the cinch and the other hand to pull on an end or wristband. The operation utilizing two hands may make it more difficult for a person to secure the indicium to herself without assistance, but it is nevertheless anticipated and may provide other benefits like ease of manufacture. In one implementation, both ends of the wristband may be threaded through the coupler 312 and then fixed securely to the cap. The coupler 312 may be housed in the cap. To tighten the wristband, the user may place the wristband around a person's wrist and then use one hand to hold the cap 304 and the other hand to tighten the coupler 312.

FIG. 3B illustrates an example of a securing mechanism 300B with a wristband formed between the coupler 312 and the cap 304. The securing mechanism 300B includes a coupler 312 that is a cinch. The coupler 312 includes the band 314 threaded through twice. The band 314 is coupled to the cap 304 at both ends. The securing mechanism 300B receives an anatomical feature in a wristband formed between the coupler 312 and the cap 304. The coupler 312 may be advanced proximally towards the cap 304 to secure the anatomical feature. The band 314 includes a loop at a distal end to which an endpiece 340 is coupled. In this implementation, the coupler 312 may be stored against the endpiece 340 prior to the decoupling of the indicium from the dispensing device. The securing mechanism 300B may be an implementation of a non-self-cinch concept.

FIG. 3C illustrates another example of a securing mechanism 300C with a wristband at a distal end of a band 314 relative to the cap 304. The securing mechanism 300C includes a coupler that is a cinch. The coupler 312 has the band 314 threaded through once. The band is coupled to the cap 304 at one end of the band 314 and to the coupler 312 at another end of the band 314. The securing mechanism 300C receives an anatomical feature in a wristband formed between the coupler 312 and the cap 304. The coupler 312 can be advanced distally from the cap 304 to secure the anatomical feature. In this implementation, pulling the cap 304 away from the anatomical feature may tighten the wristband to secure the anatomical feature. The securing mechanism 300C may comprise an implementation of a self-cinch concept. In an implementation, the wristband loops onto itself instead of utilizing a separate coupler 312 component. As such, the coupler 312 may include securing features such as a pawl that may engage features of the band 314 (e.g., teeth, threads, or the like). In this regard, the coupler 312 and band 314 may act as a zip tie that irreversibly (or irreversible without evidently damaging the band 314) engages an anatomical feature. The threaded part of the wristband may be hidden underneath the label during storage when still coupled to the dispensing device.

FIG. 3D illustrates an example of a securing mechanism 300D in which a portion of a band 314 is coupled to another portion of a band 314. The securing mechanism 300D includes a coupler 312 that attaches one part of a band 314 with another part of the same band 314 or of a different band. Such coupler 312 may comprise an adhesive strip suitable to adhere to another portion of the band 314. The anatomical feature is introduced to a wristband formed between the bands 314 or portions of the same band 314, and the bands 314 or same band 314 are coupled at a location distal from the introduced anatomical feature to secure the anatomical feature. FIG. 3D may be an implementation of a no-cinch system. That is, a coupling can be performed without cinching. In one implementation, the wristband may attach securely to the cap with the other ends free. In one implementation, one strap is shorter and includes an adhesive portion, such as with a removable backing. The end may be completely covered by the label before decoupling from the dispensing device, and the other strap may be longer and protrude from the end of the label for a user to grab and arm the dispensing device.

In the implementation in which the bands 314 are different, both may be coupled at one end to a cap 304. At least the second band includes a coupler 312 to couple the other band 314. The coupler 312 may comprise an adhesive patch. In an implementation in which the same band 314 is coupled to itself, the band 314 may be threaded through the cap 304 and may adjust the distance of the coupling from the cap 304 by threading the band 314 through the cap and adjusting the relative length of the portions of the band 314 before coupling of the portions of the band 314. This adjustability may allow for a dynamic fitting of the securing mechanism 300D to the anatomical feature.

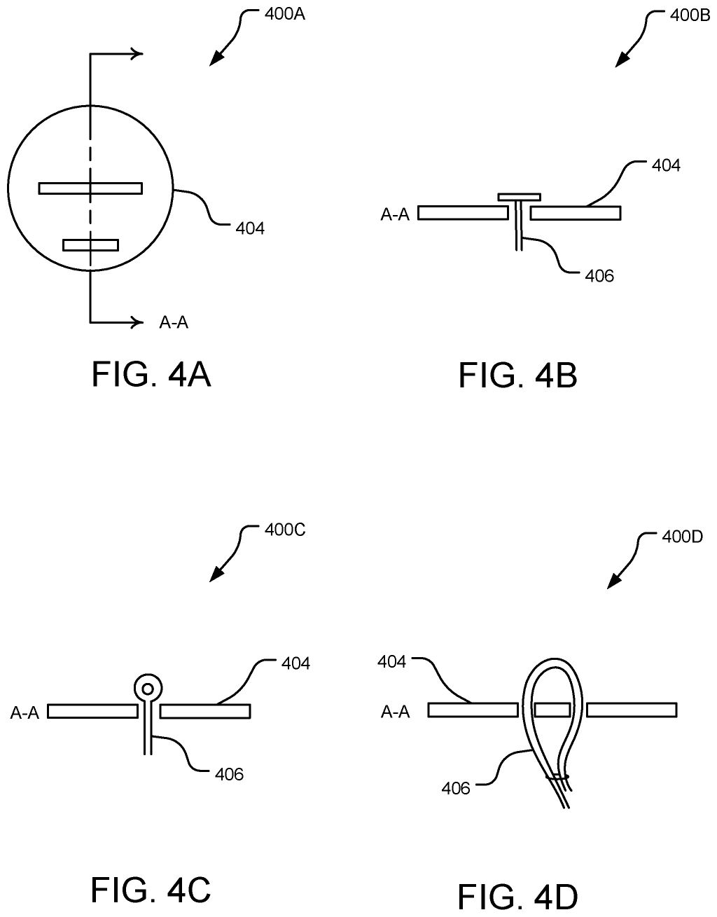

FIGS. 4A-4D illustrate examples of systems 400A-400D for coupling the cap 404 to the indicium 406. The system described may be characterized as a simple slot or bar incorporated into the cap with other elements. The cap 404 and indicium 406 may be implementations of cap 104 and indicium 106, respectively. Specifically, FIG. 4A illustrates a top view of an example of a system 400A with a cap 404 including holes. The holes may be for threading a band through or for receiving other elements to couple the cap 404 to the indicium 406. The arrows denoted “A-A” in FIG. 4A are drawn to represent that FIG. 4A shows a top perspective relative to the side perspective of FIGS. 4B-4D.

FIG. 4B illustrates a side view of an example of a system 400B for coupling the cap 404 to the indicium 406. In this implementation, the indicium 406 may include a T feature sewn into a lanyard of the indicium 406 that secures the lanyard to the cap 404.

FIG. 4C illustrates a side view of a variant system 400C for coupling the cap 404 to the indicium 406. In this implementation, the indicium 406 includes a rod or feature rolled into the lanyard that provides interference to prevent pull-through of the lanyard through the cap 404.

FIG. 4D illustrates still another side view of an example of a system 400D for coupling the cap 404 to the indicium 406. In this implementation, the indicium 406 includes a lanyard that is fed through a slot of the cap 404 and is secured at a loop by a coupling element, such as a clamp.

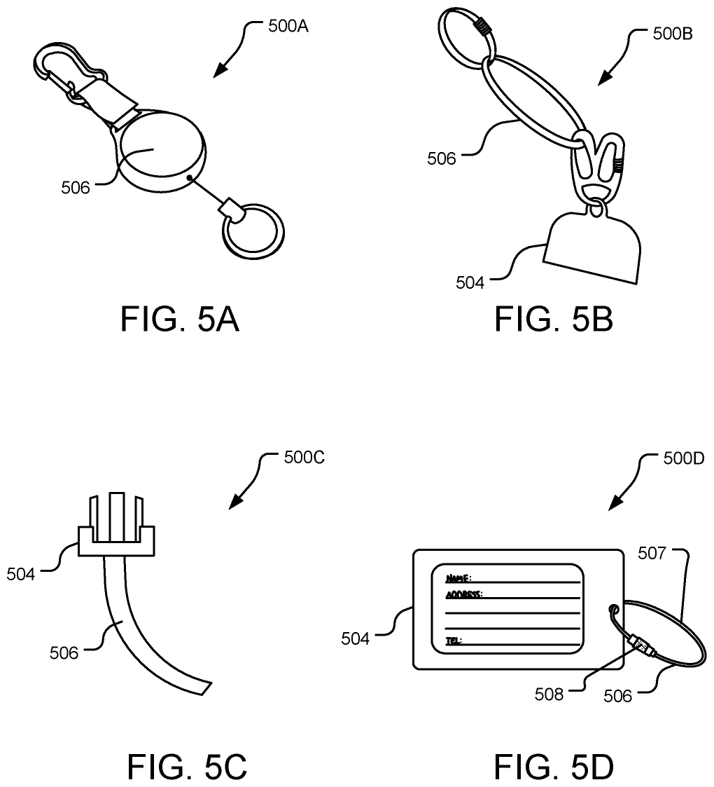

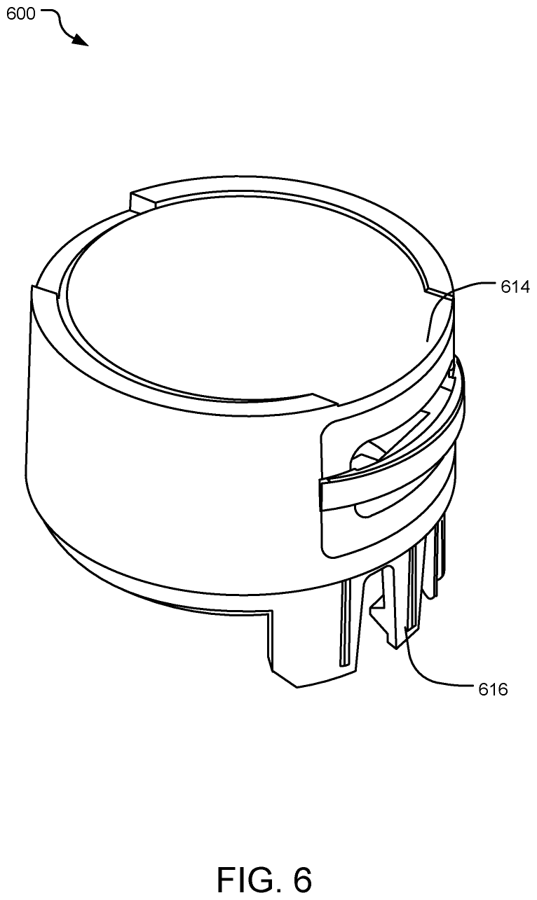

FIGS. 5A-5D illustrate further implementations of systems 500A-500D for coupling a cap to indicium. Cap 504 and indicium 506 may be implementations of cap 104 and indicium 106, respectively. Specifically, FIG. 5A illustrates an example of a system 500A with a retractable lanyard. The retractable lanyard may be an element of an indicium 506 which may be coupled to a cap 504.

FIG. 5B illustrates an example of a system 500B with a carabiner-type loop directly coupled to the cap that can be used with no lanyard. In some variations, the system 500B may be modified to include a lanyard and hook. The cap 504 may be coupled to a carabiner of an indicium 506.

FIG. 5C illustrates an example of a system 500C with an indicium 506 that includes a lanyard integrated into a cap 504 that is a plastic cover, such as a thermoplastic-polyurethane aseptic cover. The integration may involve welding or co-molding.

FIG. 5D illustrates an example of a system 500D with indicium 506 comprising a cable 507 with a threaded connector 508. The indicium 506 may be directly assembled onto or threaded through the cap 504.

FIG. 6 illustrates an example of a cap 600. The cap 600 may be an implementation of the cap 104. The cap 600 may include a capping element 614 and a coupling element 616. The capping element is the element of the cap 600 that caps a dispensing device. The capping element 614 can be used to restrict access to a dispensing device and can preclude dispensing of a substance contained in the dispensing device until the cap 600 is removed. The coupling element 616 is an element of the cap 600 that can be used to couple the cap 600 to the dispensing device. The coupling element 616 may be specifically adapted to engage elements of the dispensing device such that the dispensing device cannot dispense its contents until the coupling element 616 is disengaged from elements of the dispensing device. In some implementations, decoupling the coupling element 616 from elements of the dispensing device may activate or arm the dispensing device. The activation or arming of the dispensing device may allow the dispensing device to dispense dispensable substances contained in the dispensing device.

FIGS. 7A-7E illustrate an example arrangement of a dispensing device 700 of the present disclosure teaching the basic principles of its construction and operation. The dispensing device 700 may be an implementation of the dispensing device 102. In an implementation, the dispensing device 700 may be one or more of an autoinjector and an administration device, and these terms may be used interchangeably for the purposes of describing the dispensing device 700. The dispensing device 700 comprises a shell 702 (hereafter, by itself or in combination with other autoinjector elements, sometimes referred to as the autoinjector housing or autoinjector body), and a needle housing 712, together defining the external shell of the dispensing device 700. The dispensing device 700 has a generally elongated cylindrical form comprising a proximal end (hereafter sometimes referred to as the proximal injection end) 701 comprising a needle port 703, and a distal end, where the dispensing device 700 may be hand grasped during the injection procedure. A movable piston assembly (also referred to as the administration assembly 720) separates the interior volume of the autoinjector into a pressure chamber 704 and a sterile needle housing 712. The administration assembly 720 comprises a piston 721, a dispensable substance (e.g., a beneficial agent) package assembly 730 joined to its distal end, within the pressure chamber 704, and a movable needle assembly 740 joined at its proximal end, within the needle housing 712. The needle assembly 740 comprises a needle 741. The needle 741 includes a proximal sharp end, a distal sharp end, and a needle hub 742. The needle 741 is movable between a first pre-use position and a second injection position, as will be detailed in the FIGS. 7A-7E.

The package assembly 730 comprises a first wall 731 and a second wall 732 joined along their peripheral edges in a permanent seal fashion. The first wall 731 and the second wall 732 are further joined across the package to form a first frangible seal 736 defining a first compartment 734 adjacent to a second compartment 735. A second frangible seal 737 separates between the compartments 734-735 and a fitment 738 that is joined to the piston 721. The first compartment 734 and the second compartment 735 may be filled with at least one dispensable substance (e.g., a beneficial agent) or constituents thereof. The fitment 738 comprises a membrane 739 confronting the distal sharp end of the needle 741. A spring 714 biases the piston assembly toward the pressure chamber 704. An adhesive label 715 seals over the needle port 703 to maintain the needle 741 sterile until the time of use. In implementations, the dispensing device 700, the first compartment 734, and the second compartment 735 can be merged, by breaking the first frangible seal 736, allowing the at least first dispensable substance constituent in the first compartment 734 and at least a second constituent in the second compartment 735 to mix and or interact. The package assembly 730 may comprise more than two mergeable compartments. In an implementation, a second frangible seal 737 of the package assembly 730 can be ruptured, establishing fluid communication between the dispensable substance(s) and the fitment 738.

Pressure may be introduced into the pressure chamber 704 in the autoinjector body. The pressure is introduced from a pressure source that, when opened, creates pressure in the autoinjector body. When the pressure surpasses a first threshold value, the pressure causes the piston to overcome the spring 713 force displacing the administration assembly 720 toward the proximal injection end 701. The needle assembly 740 moves along with the piston until the needle hub 742 stops against the bottom of the needle housing 712. During usage of the dispensing device 700, in this configuration, it is held at the distal end 719 while the proximal injection end 701 is depressed against an injection site of a subject, such that the proximal end of the needle 741 is inserted into the subject. An arrangement of the pressurizing mechanism of the pressure chamber 704 is shown in FIGS. 7A-7E. After pressurization, the administration assembly 720 may continue its displacement toward the proximal injection end 701, while the needle assembly 740 is stationary, causing the back end of the needle to pierce the membrane 739 of the fitment 738, thereby establishing fluid communication between the package assembly 730 and the needle 741. The pressure in the pressure chamber 704 causes the first wall 731 to collapse and deliver the dispensable substance(s) through the needle 741. The pressure in the pressure chamber 704 is gradually reduced by releasing the gas within through a bleeding arrangement (not shown). After the pressure in the pressure chamber 704 has dropped to a limit, the spring 713 can displace the administration assembly 720 toward the distal end 719 of the actuation assembly 770 until the needle is concealed again in the needle housing 712.

Specifically, FIG. 7A illustrates an example of an arrangement of a dispensing device 700. In an implementation, the dispensing device includes an autoinjector body comprising a shell 702 and a needle housing 712, which together form the exterior of the dispensing device 700. An administration assembly 720 is disposed in the autoinjector body (hereafter can be referred to as the autoinjector housing) and comprises a piston 721, accommodating a package assembly 730 at its distal side and a needle assembly 740 at its proximal side. An actuation assembly 770 is disposed in the pressure chamber 704, comprising a support structure 773, which holds in alignment a pressurized cartridge 771 in a confronting position to a piercing pin 772. The support structure 773 further accommodates a compression panel 774 for manipulating the compartment of the administration assembly 790 to merge. FIG. 7B illustrates a cross-section of an example of a dispensing device 700 in the first autoinjector state (hereafter can be referred to as the pre-use configuration). The compression panel 774 confronts the first compartment 734 on its first side and leans against a cam 781 at its second side. The cam 781 is fixed to the shell 702. A latch feature 782 is also fixed to the shell 702. The pin's 772 sharp end faces the pierceable region of the pressurized cartridge 771 and is free to move along its axis. However, in this configuration, nothing can apply an axial force to the pin 772, and hence an accidental piercing of the cartridge 771 is not possible.

Arming trigger 756 is an element that causes the dispensing device 700 to transition to an armed state. While illustrated as a strap, the arming trigger 756 may be an element of a removable cap. Removal of the cap can cause the arming trigger to cause the dispensing device 700 to transition to an armed state. The cap may be removed when an indicium or strap is removed, or an indicium may be an element of the cap. In an implementation, the cap can be removed from the dispensing device 700 by pulling a strap (e.g., a lanyard or wristband), perhaps pulling the strap through a label. In an implementation, when a label is torn, it is torn at perforations in the label, the label including a removable portion that is coupled to a removable strap. The removal of the strap may facilitate the removal of the cap. The removal of the cap may arm the dispensing device 700.

FIG. 7C illustrates an example of an autoinjector in a second autoinjector state and a mixing autoinjector state wherein the shell 702 is extended away from the needle housing 712, causing the cam 781 to push the compression panel 774, which in return depresses the administration assembly 790 causing its compartments to merge and establish fluid communication with the fitment 738. The support structure 773 holds the pin 772 from moving upward while the latch feature 782 moves along with the shell 702, slides over the pin's 772 shaft, and engages with a detent ridge 775 of the pin 772. At this point, the pin 772 is retained to the shell 702. A slot 791 in the shell 702 allows connecting an anchoring link (hereafter sometimes referred to as the anchoring link) such as a strap or other devices to facilitate pulling the shell 702 to the extended position.

FIG. 7D illustrates an example of an autoinjector in an armed state. In FIG. 7D, the shell 702 is moved down, carrying along the pin 772, which pierces the pierceable region of the pressurized cartridge 771, thereby pressurizing the pressure chamber 704. The pressurized cartridge 771 may contain carbon dioxide, nitrogen, argon, or other gasses or supercritical fluids. When the pressure in the pressure chamber 704 exceeds a first threshold value, the force it exerts on the piston 705 overcomes the spring 713 force, moving the piston 721 toward the proximal injection end 701. The needle assembly 740 moves with the piston 721 until it is stopped by the needle housing 712 floor, at which point the needle 741 is fully extended. With the needle assembly 740 now stationary, the pressure in the pressure chamber 704 continues to move the piston 721 down, resulting in a relative movement between the needle assembly 740 and the piston 721, causing the distal end of the needle 741 to pierce the package assembly 730 and establish fluid communication with the dispensable substance. With the pressure chamber 704 still pressurized, the dispensable substance is squeezed out of the package assembly 730 and administered via the needle 741 to a subject. In one arrangement, the position of the cam 781 relative to the compression panel 774 and the position of the latch feature 782 relative to the detent ridge 775 are such that a first move of the needle housing 712 changes the dispensing device 700 to the mixing dispensing device 700 state, where the compartments of the package assembly 730 are merged, and a subsequent second move moves the dispensing device 700 to the third autoinjector state (hereafter sometimes referred to as the armed configuration). This arrangement is particularly beneficial where the dispensable substance is ready for injection only after a certain time from mixing, such that the device can be kept in a safe, unarmed state during mixing.

FIG. 7E illustrates an example of an dispensing device 700 in a manufacturing configuration. The needle housing 712 assembly is sterile and ready to fill at the filling site. Special access regions 793 in the package assembly 730 allow the filling machine to fill the package and subsequently seal the channels 792 connecting the access regions 793 and the compartment 734 and the compartment 735 to prevent fluid communication there between. When filling is completed, the access regions 793 may be removed, and the shell 702 is installed on the needle housing assembly. In an implementation, in the arrangement of FIG. 7A-7D, the dispensing device 700 comprises: (a) a shell 702 including a proximal injection end 701, (b) an administration assembly 790, comprising the administration assembly 720, the actuation assembly 770, and the needle housing 712, disposed within the shell 702, the administration assembly 790 including a delivery end proximate to the proximal injection end 701 through which a dispensable or administrable substance housed within the administration assembly 790 is capable of being expelled; when in the dispensing device 700 state illustrated in FIGS. 7A-7B (e.g., unarmed state), the administration assembly 790 is rendered incapable of expelling the dispensable substance; and when in the dispensing device 700 state (e.g., armed state) illustrated in FIG. 7C, the administration assembly 790 is rendered capable of expelling the dispensable substance. The administration assembly 790 comprises a first compartment 734 for storing at least a first constituent of the dispensable substance and at least a second compartment 735 for storing at least a second constituent of the dispensable substance separate from the first constituent; and wherein at the autoinjector state illustrated in FIG. 7C, the first compartment 734 and the at least second compartment 735 are merged.

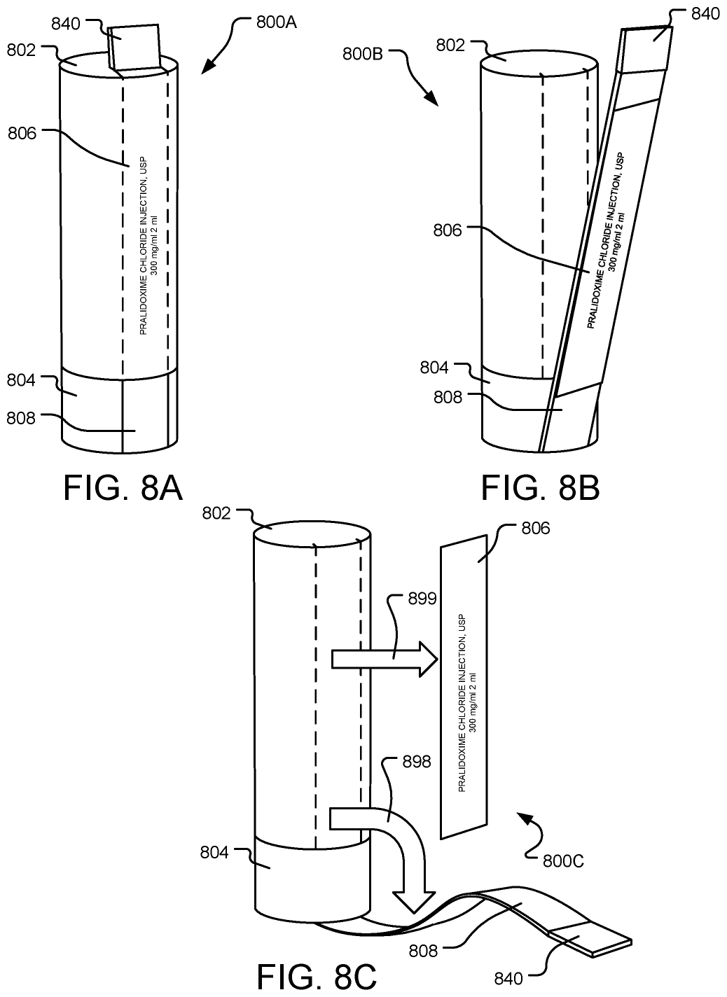

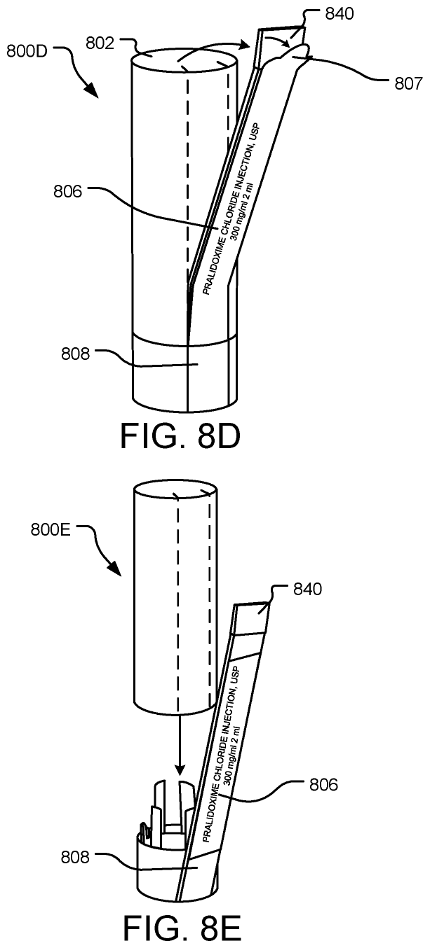

FIGS. 8A-8E illustrate examples of dispensing device systems 800A-800E with dispensing devices 802 that are armed by tearing through a label. The systems 800A-800E include a dispensing device 802, a cap 804, an indicium 806, a securing mechanism 808, and an endpiece 840. The securing mechanisms 808 (e.g., a lanyard) may include removable portions of the labels (e.g., labels adhered to or integral to the dispensing devices 802) adapted to be torn for arming the dispensing devices 802. Specifically, FIG. 8A illustrates an example dispensing device system 800A with a dispensing device 802 in a prearmed state before the tearing of a label. The indicium 806 may be a removable portion of the label. In the illustrated implementation, the indicium 806 is coupled to the label by perforations indicated by dashed lines. The indicium 806 is shown with a generic product label containing the name of a medical agent for purposes of demonstration, but the indicium may include, without limitation, identifying information associated with one or more of a patient, the dispensing device 802, and a dispensable substance contained by the dispensing device 802.

FIG. 8B illustrates an example dispensing device system 800B with a dispensing device 802 on which a securing mechanism 808 has torn through a label comprising the indicium 806. The indicium 806 is pulled away from the dispensing device 802 by pulling on an endpiece 840. The pulling causes the securing mechanism 808 to tear through a label at perforations, but the indicium 806 remains coupled to the securing mechanism 808. Pulling the securing mechanism 808 further may cause the cap 804 to be removed and may further cause the dispensing device 802 to transition to an armed state.

FIG. 8C illustrates an exploded view of an example dispensing device system 800C. Specifically, the indicium 806 is illustrated fully separated from the securing mechanism 808 and the dispensing device 802. Arrow 899 indicates that the indicium 806 can be removed at the perforations to make a strip. In some implementations, this strip may be coupled to the securing mechanism 808 such that the indicium 806, when removed from the label on the dispensing device 802, remains coupled to the securing mechanism 808. Arrow 898 indicates that the securing mechanism 808 can be removed from the dispensing device 802.

FIG. 8D illustrates an example dispensing device system 800D with a dispensing device 802 with similarities to the example of FIG. 8C. In the dispensing device system 800D, a securing mechanism 808 is shown in the process of tearing through a label comprising the indicium 806. The securing mechanism 808 (e.g., a band) and the indicium 806 are pulled away from the dispensing device 802 in some implementations by pulling on an endpiece 840. The pulling causes the securing mechanism 808 to tear through a label at perforations, but the indicium 806 remains coupled to the securing mechanism 808. The securing mechanism 808 and the indicium 806 are shown joined above the point at which the securing mechanism 808 has torn through the label. The indicium 806 further comprises a tab 807, which may be pulled to separate the indicium 806 from the securing mechanism 808. Pulling the securing mechanism 808 further may cause the cap 804 to be removed, which may further cause the dispensing device 802 to transition to an armed state.

FIG. 8E illustrates an example dispensing device system 800E in a fully torn and deployed configuration. In the fully torn and deployed configuration, the cap is fully decoupled from the dispensing device, and the indicium comprises an integral part of the band comprising the securing mechanism. In an alternative implementation, the indicium 806 may be coupled directly to the cap 804 instead of or in addition to the securing mechanism 808. The securing mechanism 808 may additionally or alternatively tear through parts of the label in implementations where the indicium 806 is coupled to the cap 804.

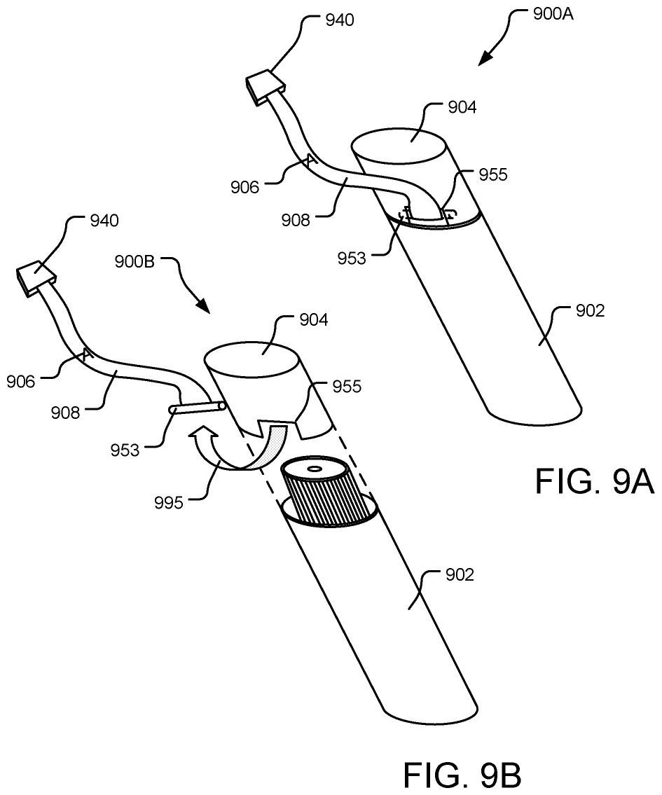

FIGS. 9A and 9B illustrate examples of states 900A-900B of an example dispensing device system. Specifically, FIG. 9A illustrates an example of a dispensing device 902 in an unarmed state 900A in which an indicium 906 is coupled by a cap 904 to the dispensing device. FIG. 9B illustrates an example of a dispensing device 902 in an armed state 900B in which the cap 904 is removed, decoupling the indicium 906 from the dispensing device 902. The dispensing device 902 may be in an unarmed state 900A or an armed state 900B. In the illustrated implementation, decoupling the cap 904 from the dispensing device 902 facilitates decoupling of the indicium from the cap 904. In the illustrated implementation, the dispensing device includes a slot 955 and a restricting element 953, such as a rod or bar. The restricting element 953 is coupled to a securing mechanism 908. In the unarmed state 900A, the securing mechanism 908 is coupled to the dispensing device 902 by a combination of the slot 955 and the restricting element 953. The restricting element 953 is large enough in relevant dimensions to prevent removal from the slot 955. Although, as illustrated, the cap 904 comprises the slot 955, the slot 955 may be a feature of any of the cap 904, a body of the dispensing device 902, or both.

The securing mechanism 908 may be further coupled to an endpiece 940. Pulling the securing mechanism 908 and/or endpiece 940 may result in a removal of the cap 904 from the dispensing device 902. The removal of the cap 904 may arm the dispensing device 902. The removal of the cap may expose a side of the slot 955 and allow the restricting element 953 to slide out of an opening in the now open slot 955, as indicated by arrow 995. Before the side of the slot 955 is exposed, the restricting element 953 may be restricted from being removed from the slot 955 until the cap 904 is removed from the dispensing device 902.

In the illustrated implementation, an indicium 906 is coupled to (or located on) the securing mechanism 908. Although not illustrated, the dispensing device 902 may further include a label on the body of the dispensing device to which the indicium 906 is initially coupled in the unarmed state 900A and through which the securing mechanism 908 tears (such as at perforations) to remove the securing mechanism 908 from the body of the dispensing device 902. In one implementation, the indicium 906 may be only removable when the cap 904 has been removed from the dispensing device 902 (perhaps only removable when the dispensing device 902 is in the armed state 900B). In the illustrated implementation, the indicium 906 may only be decouplable if the cap 904 is decoupled from the dispensing device.

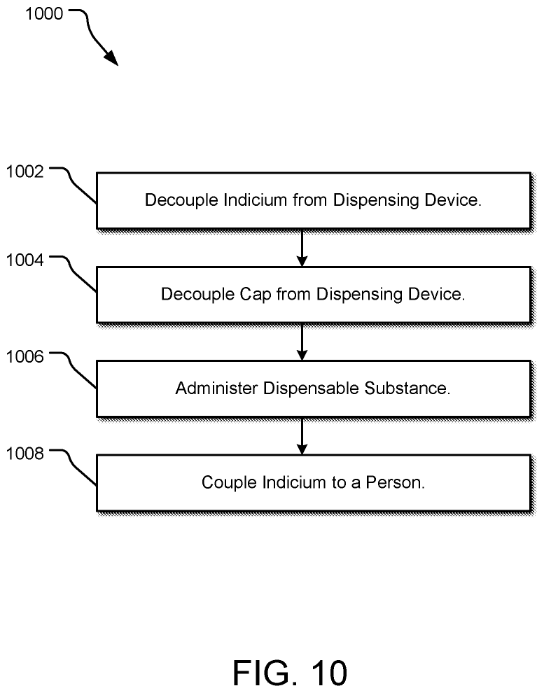

FIG. 10 illustrates a flowchart of an exemplary method 1000 of tracking the use of a dispensing device, including a decouplable indicium. Decoupling operation 1002 decouples an indicium from a dispensing device. Implementations are contemplated in which this involves tearing the indicium away from a perforated coupling between and/or through a label covering the indicium and the dispensing device. In an implementation, decoupling operation 1002 decouples the indicium and/or a securing mechanism coupled to the indicium directly from the dispensing device even without decoupling the cap from the dispensing device. The decoupling operation 1002 may involve tearing through or otherwise removing a label that held coupled one or more of the indicia and securing mechanism to the dispensing device. The removal or tearing may reveal a clear shell of the dispensing device. Decoupling operation 1004 decouples a cap from the dispensing device. The cap of a dispensing device may cover dispensing elements of the dispensing device when the cap is coupled to the dispensing device. Decoupling the cap from the dispensing device may expose dispensing elements of the dispensing device and may further activate or arm dispensing elements of the dispensing device to allow the dispensing device to dispense a dispensable substance. The dispensing or administration of the dispensable substance may involve injecting drug fluids into a patient, as with an autoinjector. While the indicium may be initially coupled to the dispensing device via the cap, in some implementations, the decoupling operation 1004 may decouple the cap from a different portion of the dispensing device than the decoupling operation 1002. For example, the cap may be decoupled or removed from an end of the dispensing device (e.g., an end with administration elements to dispense the beneficial agent and/or arming elements to arm the dispensing device), and the indicium and/or the securing mechanism may be coupled to a surface between ends of the dispensing device.

In implementations, the decoupling operation 1002 and the decoupling operation 1004 are conducted in different orders or are conducted substantially contemporaneously. For example, in implementations, the decoupling operation 1002 may be conducted before the decoupling operation 1004, as illustrated. In implementations, the indicium and/or the securing mechanism may be coupled between the ends of a dispensing device under tension, such that decoupling operation 1002 causes arming of the dispensing device and the decoupling operation 1004.

In other implementations, the decoupling operation 1002 may be conducted before the decoupling operation 1004. For example, the cap may be decoupled from the dispensing device in the decoupling operation 1004 and may be used as a grip to decouple the indicium in the decoupling operation 1002 after decoupling operation 1004.

An administering operation 1006 administers a dispensable substance to a patient from a dispensing device. The administering may be facilitated by one or more of the decoupling operations 1002 and/or 1004 allowing access to and/or arming (e.g., transitioning the dispensing device from a safe configuration to an armed configuration) the administering or dispensing elements of the dispensing device. In an implementation, the shell of the dispensing device includes a clear portion that is exposed after the decoupling operations 1002 and 1004 such that a device user can see whether the drug has been administered through the clear shell.

Coupling operation 1008 couples an indicium to a person. Coupling operation 1008 allows information provided in the indicium to be tracked from the dispensing device to the patient. The indicium may be coupled to any securable feature, including an anatomical feature or appendage of a person. The indicium may contain information, for instance, identifying information associated with one or more of the patients, the dispensing device, and the dispensable substance contained by the dispensing device. The indicium may be coupled to the person using a securing mechanism that secures the indicium to the person. A variety of implementations are contemplated with respect to the securing mechanism. Coupling operation 1008 may involve inserting an anatomical feature of the person into a band of the securing mechanism.

In an implementation, coupling operation 1008 couples the indicium to a securable feature by advancing a cinch distally from a cap of a securing mechanism. For instance, the securing mechanism may include a band and a cinch coupled to the band. The cinch may be irreversibly advanceable along at least a portion of the band. The cinch may be situated on the band relative to the cap, such that pulling the cap when the band is engaged to an anatomical feature of a person tightens the band about an anatomical feature of the person. In one implementation, the band is coupled at both a first end of the band and a second end of the band to the cap, forming a band loop. The band may be threaded twice through the cinch. The securing mechanism may receive an anatomical feature of the person between the cinch and the band loop. The securing mechanism may then be secured to the anatomical feature by irreversibly advancing the cinch distally from the cap.

In an implementation, coupling operation 1008 couples the indicium to a securable feature by advancing the cinch of a securing mechanism proximally towards a cap along a band of the securing mechanism. In this implementation in which the band is coupled at both a first end of the band and a second end of the band to the cap, forming a band loop, the securing mechanism receives an anatomical feature of a person between the cinch and the cap. The securing mechanism may then secure the anatomical feature by irreversibly advancing the cinch proximally towards the cap. In various implementations, coupling operation 1008 involves removing a cinch or other coupler from a cap. In other implementations, the cinch or coupler may be stored in a configuration in which the coupler is adjacent to (e.g., in contacting engagement with) an endpiece.

In still another alternative implementation, the coupling operation 1008 couples the indicium to a person by coupling two bands or band portions. In this implementation, two bands may be coupled to the cap, or a band may be threaded through the cap to effectively make two separate bands. The second of the bands may include a coupling element, for instance, an adhesive. An anatomical feature may be placed between the bands, and the adhesive on the second band may be used to couple the portion of the second band with the coupling to a portion of the first band, such that the coupled band portions are distal from the anatomical feature. This may effectively secure the anatomical feature between the coupled bands. In an implementation in which the bands are defined by a single band having been threaded through the cap, the band lengths may be adjusted by pulling the band through the cap to a desired or predefined length. This may allow for the coupling between the band portions (in this instance, on the same band) to more effectively secure the anatomical feature of the person.

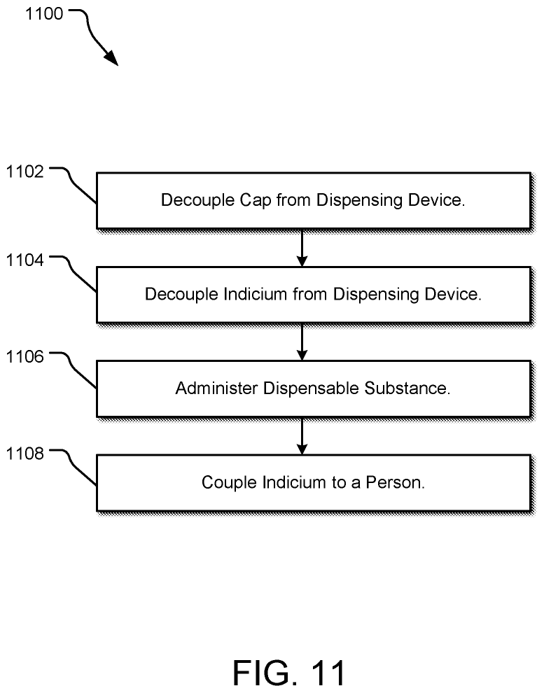

FIG. 11 illustrates an alternative implementation of the method 1100 for tracking the use of a dispensing device to dispense a beneficial agent. In this implementation, operation 1102 comprises a decoupling operation of removing the cap of the dispensing device, and operation 1104 comprises a decoupling operation of separating an indicium from the device. In implementations, the decoupling operation 1102 is an implementation of and includes features similar to operation 1004, and decoupling operation 1104 is an implementation of and includes features similar to 1002, except that the device is configured for decoupling of a decouplable cap to precede decoupling of a decouplable indicium. The administration operation 1106 is an implementation of and includes features similar to the administration operation 1006. The coupling operation 1108 is an implementation of and includes features similar to the coupling operation 1008.

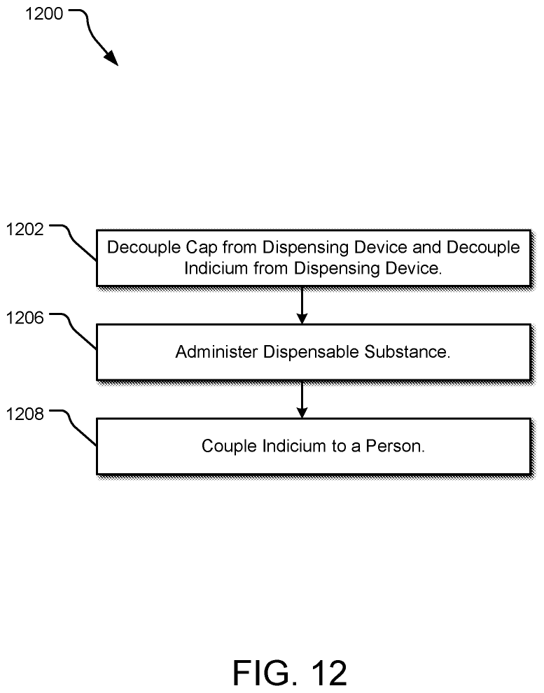

FIG. 12 illustrates an alternative implementation of the method 1200 for tracking the use of a dispensing device to dispense a beneficial agent. In this implementation, operation 1202 comprises a decoupling operation of removing the cap of the administration device and simultaneously or substantially contemporaneously decoupling an indicium from the device. In many implementations, the decoupling operation 1202 is an implementation of and includes features similar to the features of operations 1004 and 1002, except that the device is configured for the decoupling operations to comprise a single operation. The administration operation 1206 is an implementation of and includes features similar to the administration operation 1006. The coupling operation 1208 is an implementation of and includes features similar to the coupling operation 1008.

While this specification contains many specific implementation details, these should not be construed as limitations on the scope of any inventions or of what may be claimed, but rather as descriptions of features specific to particular embodiments of the particular technology described. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order or that all illustrated operations be performed to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As used herein, a beneficial agent dispensing device system may refer interchangeably to an administration device system and/or a substance dispensing system. As used herein, a dispensing device may refer interchangeably to an administration device. As used herein, a beneficial agent may refer interchangeably to a dispensable substance.

An example beneficial agent dispensing device system is provided. The beneficial agent dispensing system includes a dispensing device containing a beneficial agent, an indicium coupled to and selectively decouplable from the dispensing device, the indicium indicating information related to the beneficial agent, and a securing mechanism coupled to the indicium and configurable to secure the indicium to a securable feature.

Another example beneficial agent dispensing device system of any preceding system is provided, wherein the dispensing device includes an autoinjector.

Another example beneficial agent dispensing device system of any preceding system is provided, wherein the dispensing device is operable to administer the beneficial agent to a patient.

Another example beneficial agent dispensing device system of any preceding system is provided, wherein the indicium provides an indication that the beneficial agent has been administered to a patient.

Another example beneficial agent dispensing device system of any preceding system is provided, wherein the securing mechanism is a non-reversible attachment.

Another example beneficial agent dispensing device system of any preceding system is provided, wherein the dispensing device is adapted to transition from a safe configuration in which the dispensing device prevents dispensing of the beneficial agent to an armed configuration in which the dispensing device allows dispensing of the beneficial agent, and wherein the indicium is removable from the dispensing device in the armed configuration.

Another example beneficial agent dispensing device system of any preceding system is provided, wherein decoupling the indicium from the dispensing device transitions the dispensing device from a safe configuration in which the dispensing device prevents dispensing of the beneficial agent to an armed configuration in which the dispensing device allows dispensing of the beneficial agent.

Another example beneficial agent dispensing device system of any preceding system is provided, the beneficial agent dispensing device system further including a cap coupled to and decouplable from the dispensing device, wherein the indicium is coupled to the cap, and wherein the dispensing device is adapted to transition to the armed configuration in response to displacement of the cap.

Another example beneficial agent dispensing device system of any preceding system is provided, the beneficial agent dispensing device system further including a label configured to tear when the indicium is decoupled from the dispensing device.

Another example beneficial agent dispensing device system of any preceding system is provided, the beneficial agent dispensing device system further including a label including the indicium.

Another example beneficial agent dispensing device system of any preceding system is provided, wherein the securing mechanism includes a band adapted for selective engagement with a securable feature.

Another example beneficial agent dispensing device system of any preceding system is provided, wherein the securing mechanism includes a cinch, the cinch being irreversibly advanceable along a portion of a band, including the indicium.

Another example beneficial agent dispensing device system of any preceding system is provided, the beneficial agent dispensing device system further including a cap coupled to the dispensing device, wherein the cinch is coupled to the cap.

Another example beneficial agent dispensing device system of any preceding system is provided, wherein the securing mechanism is configured to secure the indicium to a securable feature, and wherein motion of a cap away from the securable feature advances the cinch along a portion of the band.

Another example beneficial agent dispensing device system of any preceding system is provided, the beneficial agent dispensing device system further including a cap selectively decouplable from the dispensing device, wherein the band is coupled at a first end of the band and a second end of the band to the cap, the band threaded twice through the cinch forming a band loop, the securing mechanism configured to receive a securable feature between the cinch and the band loop, the securing mechanism configured to secure the indicium to the securable feature by the cinch being irreversibly advanced distally from the cap.

Another example beneficial agent dispensing device system of any preceding system is provided, the beneficial agent dispensing device system further including a cap selectively decouplable from the dispensing device, wherein the band is coupled at both a first end of the band and a second end of the band to the cap, the band threaded twice through the cinch, the securing mechanism configured to receive a securable feature between the cinch and the cap, the securing mechanism configured to secure the indicium to the securable feature by the cinch being irreversibly advanced proximally towards the cap.

Another example beneficial agent dispensing device system of any preceding system is provided, the beneficial agent dispensing device system further including a cap selectively decouplable from the dispensing device, wherein the securing mechanism further includes an endpiece coupled to the band distally from the cap.

Another example beneficial agent dispensing device system of any preceding system is provided, the beneficial agent dispensing device system further including a cap coupled to the dispensing device, wherein the band is coupled at a first end of the band to the cap and a second end of the band to the cinch, the band threaded once through the cinch forming a band loop, the securing mechanism to receive a securable feature between the cinch and the band loop, the securing mechanism securing to the securable feature by irreversibly advancing the cinch distally from the cap.

Another example beneficial agent dispensing device system of any preceding system is provided, wherein the indicium includes an active timing element.

Another example beneficial agent dispensing device system of any preceding system is provided, wherein the active timing element includes one of a pressure-sensitive element, a photosensitive element, or a chemosensitive element.

Another example beneficial agent dispensing device system of any preceding system is provided, the indicium further including a modifiable portion suitable to indicate information related to the dispensing of the beneficial agent.

Another example beneficial agent dispensing device system of any preceding system is provided, wherein the indicium includes an adhesive portion for securing the indicium to a securable feature.

Another example beneficial agent dispensing device system of any preceding system is provided, wherein the dispensing device is adapted to transition from a safe configuration in which the dispensing device prevents dispensing of the beneficial agent to an armed configuration in which the dispensing device allows dispensing of the beneficial agent, and wherein the indicium is secured to the dispensing device in the safe configuration, and the indicium includes a pull configured to displace an activation member of the dispensing device and to transition the dispensing device to the armed configuration by the displacement.

Another example beneficial agent dispensing device system of any preceding system is provided, the beneficial agent dispensing device system further including a cap selectively decouplable from the dispensing device, wherein the indicium is secured to the cap, the indicium further directly decouplable from the dispensing device at a position on the dispensing device other than the cap.

An example method for tracking a dispensable substance in a dispensing device is provided. The method includes decoupling an indicium from the dispensing device and coupling a securing mechanism attached to the indicium to a securable feature.

Another example method of any preceding method is provided, the decoupling the indicium from the dispensing device including decoupling the indicium from a direct coupling between the indicium and the dispensing device and decoupling a cap selectively decouplable from the dispensing device from a direct coupling between the cap and the dispensing device.

Another example method of any preceding method is provided, the method further including decoupling a cap from the dispensing device, responsive to the decoupling of the indicium, wherein the coupling the securing mechanism to the securable feature includes inserting a securable feature into a band coupled at both ends of the band to the cap; and tightening the band about the securable feature.

Another example method of any preceding method is provided, wherein the band is threaded twice through a cinch and wherein the tightening includes advancing the cinch towards the cap.

Another example method of any preceding method is provided, wherein the band is threaded twice through a cinch and wherein the operation of tightening includes advancing the cinch away from the cap.

Another example method of any preceding method is provided, the coupling the indicium to the securable feature including inserting a securable feature into a band coupled at one end of the band to a cap and another end of the band to a cinch, the band threaded once through the cinch, wherein the operation of tightening includes advancing the cinch away from the cap.

Another example method of any preceding method is provided, the coupling the indicium to the securable feature including inserting a securable feature between a first band coupled at a first side of the first band to a cap and a second band coupled at a first side of the second band to the cap, the second band having an adhesive portion distal from the cap and adhering the adhesive portion of the second band to a portion of the first band distal from the securable feature.

An example system for tracking a dispensable substance in a dispensing device is provided. The system includes means for decoupling an indicium from the dispensing device and means for coupling a securing mechanism attached to the indicium to a securable feature.

Another example system of any preceding system is provided, the means for decoupling the indicium from the dispensing device including means for decoupling the indicium from a direct coupling between the indicium and the dispensing device and means for decoupling a cap selectively decouplable from the dispensing device from a direct coupling between the cap and the dispensing device.

Another example system of any preceding system is provided, the system further including means for decoupling a cap from the dispensing device, responsive to the decoupling of the indicium, wherein the means for coupling the securing mechanism to the securable feature includes means for inserting a securable feature into a band coupled at both ends of the band to the cap, the system further including means for tightening the band about the securable feature.

Another example system of any preceding system is provided, wherein the band is threaded twice through a cinch and wherein the means for tightening include means for advancing the cinch towards the cap.

Another example system of any preceding system is provided, wherein the band is threaded twice through a cinch and wherein means for tightening include means for advancing the cinch away from the cap.

Another example system of any preceding system is provided, the means for coupling the indicium to the securable feature including means for inserting a securable feature into a band coupled at one end of the band to a cap and another end of the band to a cinch, the band threaded once through the cinch, wherein the means for tightening include means for advancing the cinch away from the cap.

Another example system of any preceding system is provided, the means for coupling the indicium to the securable feature including means for inserting a securable feature between a first band coupled at a first side of the first band to a cap and a second band coupled at a first side of the second band to the cap, the second band having an adhesive portion distal from the cap, the means for coupling to the indicium to the securable feature further including means for adhering the adhesive portion of the second band to a portion of the first band distal from the securable feature.

An example administration device system is provided. The administration device system includes an administration device containing an administrable substance, wherein the administration device is adapted to transition from a first configuration to a second configuration, a cap coupled to the administration device, and an indicium including information related to the administrable substance, wherein the indicium is decouplable from the administration device and wherein the decoupling of the indicium from the administration device causes the administration device to transition from the first configuration to the second configuration.

An example administration device system is provided. The administration device system includes an administration device containing an administrable substance, wherein the administration device is adapted to transition from a first configuration to a second configuration, an indicium including instructions for use of one or more of the administrable substance and the administration device, and a cap coupled to the administration device and the indicium, wherein the cap activates arming elements within the administration device when the cap is decoupled from the administration device, the arming elements operable to transition the administration device from the first configuration to the second configuration.

An example administration device system is provided. The administration device system includes an administration device containing an administrable substance, wherein the administration device is adapted to transition from a first configuration in which the administrable substance cannot be administered to a second configuration in which the administrable substance can be administered, an indicium including information related to the administrable substance, and a cap coupled to the administration device and the indicium, wherein the cap is adapted to arm arming elements within the administration device when the cap is decoupled from the administration device, the arming elements operable to transition the administration device from the first configuration to the second configuration.

An example administration device system is provided. The administration device system includes an administration device containing an administrable substance, wherein the administration device is adapted to transition from a first configuration in which the administrable substance cannot be dispensed to a second configuration in which the administrable substance can be dispensed, an indicium including information related to the administrable substance, and a cap coupled to the administration device and the indicium, wherein the cap activates arming elements within the administration device when the cap is decoupled from the administration device, the arming elements operable to transition the administration device from the first configuration to the second configuration.

An example administration device system is provided. The administration device system includes an administration device containing an administrable substance, wherein the administration device is adapted to transition from a first configuration in which the administrable substance cannot be dispensed to a second configuration in which the administrable substance can be dispensed, a cap coupled to the administration device, and an indicium including information related to the administrable substance coupled to one or more of the cap and the administration device, wherein the cap activates arming elements within the administration device when the cap is removed from the administration device to transition the administration device from the first configuration to the second configuration, and wherein decoupling the indicium from the one or more of the cap and the administration device decouples the cap from the administration device.

An example administration device system is provided. The administration device system includes an administration device containing an administrable substance, wherein the administration device is adapted to transition from a first configuration in which the administrable substance cannot be dispensed to a second configuration in which the administrable substance can be dispensed, a cap coupled to the administration device, and an indicium including information related to the administrable substance coupled to the cap, wherein decoupling the indicium from the administration device decouples the cap from the administration device and wherein the cap activates arming elements within the administration device when the cap is removed from the administration device to transition the administration device from the first configuration to the second configuration, and wherein decoupling the cap from the administration device facilitates decoupling of the indicium from the cap.

Another example administration device system of any preceding system is provided, the administration device system further including a label, wherein the indicium is integral to the label before the indicium is decoupled.

Another example administration device system of any preceding system is provided, wherein the label further includes perforations, the perforations configured to tear when the indicium is decoupled.

Another example administration device system of any preceding system is provided, the administration device system further including a band coupled to the cap and the indicium and a label that covers the band prior to decoupling the band from the administration device, wherein the band is adapted to tear through the label when the band is decoupled from the administration device.

Another example administration device system of any preceding system is provided, wherein the label includes perforations operable to guide the tearing of the band through the label.

Another example administration device system of any preceding system is provided, wherein a portion of the label remains coupled to the band after decoupling of the indicium from the administration device.

Another example administration device system of any preceding system is provided, wherein the indicium cannot be decoupled from the cap while the cap is coupled to the administration device.

Another example administration device system of any preceding system is provided, wherein the indicium includes a writable section.

Another example administration device system of any preceding system is provided, wherein the indicium includes a manually modifiable portion to manually indicate information related to the administrable substance.

Another example administration device system of any preceding system is provided, wherein the indicium includes an active timing element.

Another example administration device system of any preceding system is provided, wherein the active timing element includes one or more of a pressure-sensitive element, a photosensitive element, and a chemosensitive element.

Another example administration device system of any preceding system is provided, the administration device system further including a restricting element, wherein one or more of the cap and the administration device include a slot, the slot partially defined by the coupling of the cap to the administration device, wherein decoupling the cap from the administration device exposes a side of the slot, and wherein the restricting element is restricted from being removed from the slot until the side of the slot is exposed.

Another example administration device system of any preceding system is provided, the administration device system further including a securing mechanism coupled to the indicium, wherein the securing mechanism is a non-reversible attachment.

Another example administration device system of any preceding system is provided, wherein the securing mechanism includes a band adapted for selective engagement with an anatomical feature of a patient.

Another example administration device system of any preceding system is provided, wherein the securing mechanism includes a cinch member that is irreversibly advanceable along a portion of a band of the indicium.

Another example administration device system of any preceding system is provided, wherein the cinch is stored within the cap prior to decoupling of the cap from the administration device.

Another example administration device system of any preceding system is provided, wherein the cinch is advanced by pulling the cap away from an anatomical feature secured by the securing mechanism.

Another example administration device system of any preceding system is provided, wherein the band is coupled at both a first end of the band and a second end of the band to the cap, forming a band loop, the band threaded twice through the cinch, the securing mechanism to receive an anatomical feature of a person between the cinch and the band loop, the securing mechanism securing to the anatomical feature by irreversibly advancing the cinch distally from the cap.

Another example administration device system of any preceding system is provided, wherein the band is coupled at both a first end of the band and a second end of the band to the cap, forming a band loop, the band threaded twice through the cinch, the securing mechanism to receive an anatomical feature of a person between the cinch and the cap, the securing mechanism securing to the anatomical feature by irreversibly advancing the cinch proximally towards the cap.

Another example administration device system of any preceding system is provided, the securing mechanism further including an endpiece at a bend of a band loop located distally from the cap.

Another example administration device system of any preceding system is provided, wherein the band is coupled at a first end of the band to the cap and a second end of the band to the cinch, the band threaded once through the cinch, forming a band loop, the securing mechanism to receive an anatomical feature of a person between the cinch and the band loop, the securing mechanism securing to the anatomical feature by irreversibly advancing the cinch distally from the cap.

Another example administration device system of any preceding system is provided, wherein the cinch member is stored in the cap or adjacent to an endpiece of the band prior to decoupling of the indicium from the administration device.

Another example administration device system of any preceding system is provided, wherein the band includes an adhesive portion for securing the indicium to a patient.

Another example administration device system of any preceding system is provided, wherein the indicium is secured to the administration device in the first configuration of the administration device, and wherein the indicium is selectively removable from the administration device to provide a pull to remove the cap, causing the arming trigger to transition the administration device to an armed state the administration device prior to injection of the administrable substance to a patient.

Another example administration device system of any preceding system is provided, wherein the indicium is further directly decouplable from the administration device at a position on the administration device other than the cap.

Another example administration device system of any preceding system is provided, wherein the indicium includes a portion with instructions for use of the administration device.

An example substance dispensing system is provided. The substance dispensing system includes a dispensing device containing a dispensable substance, an indicium for providing an indication of the dispensable substance, wherein the indicium is decouplable from the dispensing device, and a securing mechanism coupled to the indicium for securing the indicium to a patient.

Another example substance dispensing system of any preceding system is provided, wherein the dispensing device includes an autoinjector.

Another example substance dispensing system of any preceding system is provided, wherein the dispensable substance is a beneficial substance and wherein the dispensing device is operable to administer the beneficial substance to the patient.

Another example substance dispensing system of any preceding system is provided, wherein the indicium further provides an indication that the substance has been administered to the patient.

Another example substance dispensing system of any preceding system is provided, wherein the securing mechanism is a non-reversible attachment.

Another example substance dispensing system of any preceding system is provided, wherein the indicium is secured to a cap of the dispensing device, wherein the dispensing device is armed for an administration of the dispensable substance by transitioning from a first configuration in which the cap is coupled to the dispensing device to a second configuration where the cap is removed from the dispensing device.

Another example substance dispensing system of any preceding system is provided, wherein the indicium includes a band adapted for selective engagement with an anatomical feature of the patient.

Another example substance dispensing system of any preceding system is provided, wherein the indicium is secured to a cap of the dispensing device that is decouplable from the dispensing device, wherein the securing mechanism includes a cinch member that is irreversibly advanceable along a portion of a band of the indicium.

Another example substance dispensing system of any preceding system is provided, wherein the cinch is stored within the cap prior to decoupling of the cap from the dispensing device.

Another example substance dispensing system of any preceding system is provided, wherein the cinch is advanced by pulling the cap away from an anatomical feature secured by the securing mechanism.

Another example substance dispensing system of any preceding system is provided, wherein the band is coupled at both a first end of the band and a second end of the band to the cap, forming a band loop, the band threaded twice through the cinch, the securing mechanism to receive an anatomical feature of the patient between the cinch and the band loop, the securing mechanism securing to the anatomical feature by irreversibly advancing the cinch distally from the cap.

Another example substance dispensing system of any preceding system is provided, wherein the band is coupled at both a first end of the band and a second end of the band to the cap, forming a band loop, the band threaded twice through the cinch, the securing mechanism to receive an anatomical feature of the patient between the cinch and the cap, the securing mechanism securing to the anatomical feature by irreversibly advancing the cinch proximally towards the cap.

Another example substance dispensing system of any preceding system is provided, the securing mechanism further including an endpiece at a bend of a band loop located distally from the cap.

Another example substance dispensing system of any preceding system is provided, wherein the band is coupled at a first end of the band to the cap and a second end of the band to the cinch, the band threaded once through the cinch, forming a band loop, the securing mechanism to receive an anatomical feature of the patient between the cinch and the band loop, the securing mechanism securing to the anatomical feature by irreversibly advancing the cinch distally from the cap.

Another example substance dispensing system of any preceding system is provided, wherein the indicium includes an adhesive portion for securing the indicium to the patient.

Another example substance dispensing system of any preceding system is provided, wherein the indicium is secured to the dispensing device in a safe configuration of the dispensing device, and wherein the indicium is selectively removable from the dispensing device to provide a pull to remove an activation member of the dispensing device to arm the dispensing device prior to injection of the dispensable substance to the patient.

Another example substance dispensing system of any preceding system is provided, wherein the indicium is secured to a cap of the dispensing device that is decouplable from the dispensing device, the indicium further directly decouplable from the dispensing device at a position on the dispensing device other than the cap.

An example method for tracking a dispensable substance in a dispensing device having a decouplable indicium is provided. The method includes decoupling the indicium from the dispensing device and coupling the indicium to an anatomical feature of a patient using a securing mechanism coupled to the decouplable indicium.

Another example method of any preceding method is provided, the decoupling the indicium from the dispensing device including decoupling a cap of the dispensing device coupled to the indicium from the dispensing device and decoupling the indicium from a direct coupling between the indicium and the dispensing device at a location on the dispensing device other than the cap.

Another example method of any preceding method is provided, the operation of coupling indicium to the anatomical feature including inserting an anatomical feature of the patient into a band coupled at both ends of the band to a cap and tightening the band about the anatomical feature.

Another example method of any preceding method is provided, wherein the band is threaded twice through a cinch and wherein the tightening includes advancing the cinch towards the cap.

Another example method of any preceding method is provided, wherein the band is threaded twice through a cinch and wherein the tightening includes advancing the cinch away from the cap.

Another example method of any preceding method is provided, the operation of coupling the indicium to the anatomical feature including inserting an anatomical feature of the person into a band coupled at one end of the band to a cap and another end of the band to a cinch, the band threaded once through the cinch, wherein the operation of tightening includes advancing the cinch away from the cap.

Another example method of any preceding method is provided, the operation of coupling the indicium to the anatomical feature including inserting an anatomical feature of the patient between a first band coupled at a first side of the first band to a cap and a second band coupled at a first side of the second band to the cap, the second band having an adhesive portion distal from the cap and adhering the adhesive portion of the second band to a portion of the first band distal from the anatomical feature.

An example system for tracking a dispensable substance in a dispensing device having a decouplable indicium is provided. The system includes means for decoupling the indicium from the dispensing device and means for coupling the indicium to an anatomical feature of a patient using a securing mechanism coupled to the decouplable indicium.

Another example system of any preceding system is provided, the means for decoupling the indicium from the dispensing device including means for decoupling a cap of the dispensing device coupled to the indicium from the dispensing device and means for decoupling the indicium from a direct coupling between the indicium and the dispensing device at a location on the dispensing device other than the cap.

Another example system of any preceding system is provided, the means for coupling indicium to the anatomical feature including means for inserting an anatomical feature of the patient into a band coupled at both ends of the band to a cap and means for tightening the band about the anatomical feature.

Another example system of any preceding system is provided, wherein the band is threaded twice through a cinch and wherein the means for tightening include means for advancing the cinch towards the cap.

Another example system of any preceding system is provided, wherein the band is threaded twice through a cinch and wherein the means for tightening includes means for advancing the cinch away from the cap.

Another example system of any preceding system is provided, the means for coupling the indicium to the anatomical feature including means for inserting an anatomical feature of the person into a band coupled at one end of the band to a cap and another end of the band to a cinch, the band threaded once through the cinch, wherein the means for tightening include means for advancing the cinch away from the cap.

Another example system of any preceding system is provided, the means for coupling the indicium to the anatomical feature including means for inserting an anatomical feature of the patient between a first band coupled at a first side of the first band to a cap and a second band coupled at a first side of the second band to the cap, the second band having an adhesive portion distal from the cap, the means for coupling the indicium to the anatomical feature further including means for adhering the adhesive portion of the second band to a portion of the first band distal from the anatomical feature.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

A number of implementations of the described technology have been described. Nevertheless, it will be understood that various modifications can be made without departing from the spirit and scope of the recited claims.

1. A beneficial agent dispensing device system, comprising:
a dispensing device containing a beneficial agent;
an indicium coupled to and selectively decouplable from the dispensing device, the indicium indicating information related to the beneficial agent; and
a securing mechanism coupled to the indicium and configurable to secure the indicium to a securable feature.

2. The beneficial agent dispensing device system of claim 1, wherein the dispensing device comprises an autoinjector.

3. (canceled)

4. The beneficial agent dispensing device system of claim 1, wherein the indicium provides an indication that the beneficial agent has been administered to a patient.

5. (canceled)

6. The beneficial agent dispensing device system of claim 1,
wherein the dispensing device is adapted to transition from a safe configuration in which the dispensing device prevents dispensing of the beneficial agent to an armed configuration in which the dispensing device allows dispensing of the beneficial agent, and
wherein the indicium is removable from the dispensing device in the armed configuration.

7. The beneficial agent dispensing device system of claim 1, wherein decoupling the indicium from the dispensing device transitions the dispensing device from a safe configuration in which the dispensing device prevents dispensing of the beneficial agent to an armed configuration in which the dispensing device allows dispensing of the beneficial agent.

8. The beneficial agent dispensing device system of claim 7, further comprising:
a cap coupled to and decouplable from the dispensing device,
wherein the indicium is coupled to the cap, and
wherein the dispensing device is adapted to transition to the armed configuration in response to displacement of the cap.

9. (canceled)

10. (canceled)

11. The beneficial agent dispensing device system of claim 1, wherein the securing mechanism comprises a band adapted for selective engagement with the securable feature.

12. The beneficial agent dispensing device system of claim 1, wherein the securing mechanism comprises a cinch, the cinch being irreversibly advanceable along a portion of a band, including the indicium.

13. The beneficial agent dispensing device system of claim 8, further comprising:
a cap coupled to the dispensing device, wherein the cinch is coupled to the cap.

14. The beneficial agent dispensing device system of claim 8, wherein the securing mechanism is configured to secure the indicium to the securable feature, and
wherein motion of a cap away from the securable feature advances the cinch along a portion of the band.

15. The beneficial agent dispensing device system of claim 8, further comprising:
a cap selectively decouplable from the dispensing device, wherein the band is coupled at a first end of the band and a second end of the band to the cap, the band threaded twice through the cinch forming a band loop, the securing mechanism configured to receive the securable feature between the cinch and the band loop, the securing mechanism configured to secure the indicium to the securable feature by the cinch being irreversibly advanced distally from the cap.

16. The beneficial agent dispensing device system of claim 8, further comprising:
a cap selectively decouplable from the dispensing device, wherein the band is coupled at both a first end of the band and a second end of the band to the cap, the band threaded twice through the cinch, the securing mechanism configured to receive the securable feature between the cinch and the cap, the securing mechanism configured to secure the indicium to the securable feature by the cinch being irreversibly advanced proximally towards the cap.

17. (canceled)

18. The beneficial agent dispensing device system of claim 12, further comprising:
a cap coupled to the dispensing device, wherein the band is coupled at a first end of the band to the cap and a second end of the band to the cinch, the band threaded once through the cinch forming a band loop, the securing mechanism to receive the securable feature between the cinch and the band loop, the securing mechanism securing to the securable feature by irreversibly advancing the cinch distally from the cap.

19. The beneficial agent dispensing device system of claim 1, wherein the indicium comprises an active timing element.

20. (canceled)

21. (canceled)

22. (canceled)

23. The beneficial agent dispensing device system of claim 1, wherein the dispensing device is adapted to transition from a safe configuration in which the dispensing device prevents dispensing of the beneficial agent to an armed configuration in which the dispensing device allows dispensing of the beneficial agent, and
wherein the indicium is secured to the dispensing device in the safe configuration, and the indicium includes a pull configured to displace an activation member of the dispensing device and to transition the dispensing device to the armed configuration by the displacement.

24. (canceled)

25. A method for tracking a dispensable substance in a dispensing device, the method comprising:
decoupling an indicium from the dispensing device; and
coupling a securing mechanism attached to the indicium to a securable feature.

26. The method of claim 16, the decoupling the indicium from the dispensing device comprising:
decoupling the indicium from a direct coupling between the indicium and the dispensing device; and
decoupling a cap selectively decouplable from the dispensing device from a direct coupling between the cap and the dispensing device.

27. The method of claim 16, further comprising:
decoupling a cap from the dispensing device, responsive to the decoupling of the indicium, wherein the coupling the securing mechanism to the securable feature includes:
inserting the securable feature into a band coupled at both ends of the band to the cap; and
tightening the band about the securable feature.

28. (canceled)

29. (canceled)

30. The method of claim 16, the coupling the indicium to the securable feature comprising:
inserting the securable feature into a band coupled at one end of the band to a cap and another end of the band to a cinch, the band threaded once through the cinch, wherein the tightening comprises advancing the cinch away from the cap.

31. The method of claim 16, the coupling the indicium to the securable feature comprising:
inserting the securable feature between a first band coupled at a first side of the first band to a cap and a second band coupled at a first side of the second band to the cap, the second band having an adhesive portion distal from the cap; and
adhering the adhesive portion of the second band to a portion of the first band distal from the securable feature.
